(19) United States
(12) Patent Application Publication
(10) Pub. No.: US 2025/0073034 A1
(43) Pub. Date: Mar. 6, 2025

(54) FIXATION SYSTEM WITH LEAFLET CAPTURE ASSESSMENT

(71) Applicant: Evalve, Inc., Santa Clara, CA (US)

(72) Inventors: Chad J. Abunassar, Alameda, CA (US); Darin Ursuliak, Lancaster, MA (US)

(73) Assignee: Evalve, Inc., Santa Clara, CA (US)

(21) Appl. No.: 18/816,445

(22) Filed: Aug. 27, 2024

Related U.S. Application Data
(60) Provisional application No. 63/579,824, filed on Aug. 31, 2023.

(51) Int. Cl. A61F 2/24

(52) U.S. Cl. CPC A61F 2/2466; A61F 2/246; A61F 2/2463; A61F 2220/0008

(57) ABSTRACT

A fixation system includes a delivery device, a fixation device, and an optical coherence tomography (OCT) catheter. The delivery device includes a shaft that defines a lumen. The fixation device includes a first clamp, a second clamp, and a center portion. The center portion is releasably connected to a distal end of the shaft of the delivery device, and the first and second clamps define respective first and second lateral extents of the fixation device. The OCT catheter includes an imaging probe that comprises a first end, a second end, and a first lens assembly that is disposed at the second end of the imaging probe. In an assembled condition of the fixation system, the imaging probe extends through the lumen and out from the second end of the shaft such that the lens assembly is positioned between the first and second lateral extents of the fixation device.

### CROSS-REFERENCE TO RELATED APPLICATIONS

This application claims the benefit of the filing date of U.S. Provisional Application No. 63/579,824, filed Aug. 31, 2023, the disclosure of which is hereby incorporated herein by reference.

### BACKGROUND OF THE DISCLOSURE

The present disclosure relates generally to medical methods, devices, and systems. In particular, the present disclosure relates to methods, devices, and systems for the endovascular, percutaneous, or minimally invasive surgical treatment of bodily tissues, such as tissue approximation or valve repair. More particularly, the present disclosure relates to repairing heart valves and venous valves, and devices and methods for assessing the quality of valve leaflet capture and approximation through minimally invasive procedures.

Surgical repair of bodily tissues often involves tissue approximation and fastening of such tissues in the approximated arrangement. When repairing valves, tissue approximation includes coapting the leaflets of the valves in a therapeutic arrangement which may then be maintained by fastening or fixing the leaflets. Such coaptation can be used to treat regurgitation which most commonly occurs in the mitral valve but is also routinely found, although less commonly, in the tricuspid valve.

Mitral valve and tricuspid valve regurgitation are characterized by retrograde flow from a ventricle of a heart through an incompetent valve into a respective atrium. During a normal cycle of heart contraction (systole), the mitral valve and tricuspid valve each act as a check valve to prevent blood from flowing back into the left atrium and right atrium, respectively. In this way, oxygenated blood is pumped into the aorta through the aortic valve from the left ventricle, and deoxygenated blood is pumped to the lungs through the pulmonary valve from the right ventricle. Valve regurgitation can significantly decrease the pumping efficiency of the heart, placing the patient at risk of severe, progressive heart failure.

Valve regurgitation, be it mitral valve regurgitation or tricuspid valve regurgitation, can result from a number of different mechanical defects in the valve or the corresponding ventricular wall. The valve leaflets, the valve chordae which connect the leaflets to the papillary muscles, the papillary muscles themselves, or the ventricular wall may be damaged or otherwise dysfunctional. Commonly, the valve annulus may be damaged, dilated, or weakened, limiting the ability of the valve to close adequately against the high pressures of the ventricles.

The most common treatments for mitral and tricuspid valve regurgitation rely on valve replacement or repair including leaflet and/or annulus remodeling, the latter generally referred to as valve annuloplasty. One technique for valve repair which relies on suturing adjacent segments of opposed valve leaflets together is referred to as the “bow-tie” or “edge-to-edge” technique. While all these techniques can be effective, they usually rely on open heart surgery where the patient's chest is opened, typically via a sternotomy, and the patient placed on cardiopulmonary bypass. The need to both open the chest and place the patient on bypass is traumatic and has associated high mortality and morbidity.

However, in some patients, an edge-to-edge repair can be achieved using a fixation device which can be installed into the heart using minimally invasive techniques. The fixation device can hold opposed valve leaflets together and may reduce valve regurgitation. One such device used to clip the anterior and posterior leaflets of the mitral valve together is the MitraClip® fixation device, sold by Abbott Vascular, Santa Clara, California, USA, and one such device used to clip opposed valve leaflets of the tricuspid valve together is the TriClip® fixation device, sold by Abbott Vascular, Santa Clara, California, USA.

### BRIEF SUMMARY OF THE DISCLOSURE

In one example of the present disclosure, a fixation system for engaging tissue of a patient includes a delivery device. The delivery device includes a shaft that defines a lumen that extends from a first end to a second end of the shaft. The system also includes an implantable fixation device that has a first clamp, a second clamp, and a center portion connected to and extending between the first and second clamps. The center is releasably connected to a distal end of the shaft of the delivery device. The first clamp defines a first lateral extent of the fixation device, and the second clamp defines a second lateral extent of the fixation device. The system further includes a first optical coherence tomography (“OCT”) catheter that is configured for cardiovascular imaging and has a first imaging probe that comprises a first end, a second end, and a first lens assembly that is disposed at the second end of the first imaging probe. The first imaging probe is configured to transmit light between the first and second ends thereof. In an assembled condition of the fixation system, the first imaging probe extends through the lumen and out from the second end of the shaft such that the first lens assembly is positioned between the first and second lateral extents of the fixation device.

Additionally, the first imaging probe may include a sheath and an optical fiber disposed within the sheath. The first lens assembly may be disposed at an end of the optical fiber. The optical fiber may be rotatable and translatable within the sheath. Also, the OCT catheter may include a flush feature that has an inlet port at the first end of the first imaging probe and an outlet port at the second end of the first imaging probe. The lens assembly may include a lens and a beam deflector. The beam deflector may be configured to deflect the light transmitted through the first imaging probe in a direction radially outwardly from a central axis of the imaging probe. The beam deflector may be configured to deflect the light at a perpendicular angle relative to the central axis.

Furthermore, the delivery device may include a handle that is connected to the first end of the shaft. The handle may have an OCT probe interface configured to receive the first imaging probe and direct it into the lumen of the shaft. The lumen may be a first lumen of a plurality of lumens. The delivery device may further include an actuator rod that extends through a second lumen of the plurality of lumens and out from the second end of the shaft where the actuator rod engages the center portion. The actuator rod may be configured to move the first and second clamps from a first position to a second position.

Continuing with this example, a first plane and a second plane may each bisect the center portion. The first plane may be orthogonal to the second plane and may intersect the first and second clamps. The second plane may be located equidistant from each of the first and second clamps. Additionally, the first lens assembly may be positioned adjacent to the center portion such that the first plane intersects the first lens assembly. Alternatively, the first lens assembly may be positioned adjacent to the center portion such that the second plane intersects the first lens assembly. The first and second planes may also define four quadrants arranged about the center portion. As another alternative, first lens assembly may be positioned adjacent to the center portion and within one of the four quadrants.

Also, the first clamp may include a first proximal element and a first distal element. The second clamp may include a second proximal element and a second distal element. The first imaging probe may extend distally along the center portion and proximally into a space between the first proximal element and the first distal element of the first clamp such that the first lens assembly is positioned within the space. The first distal element may include a crossbar that extends across at least a portion of an engagement surface thereof. The first imaging probe may extend between the crossbar and the engagement surface. The first lens assembly may be positioned at one of proximal to the crossbar and distal to the crossbar.

Additionally, the first imaging probe may extend distally along the center portion and proximally along a proximal side of the proximal element. The first imaging probe may be connected to the first side of the proximal element.

The system may also include a second OCT catheter that has a second imaging probe and a second lens assembly. The second imaging probe may extend from the shaft of the delivery device such that the second lens assembly is positioned between the first and second lateral extents in any one of the previous arrangements described with respect to the first imaging probe.

In another example of the present disclosure, a fixation system for engaging tissue of a patient includes an implantable fixation device. The implantable fixation device includes a first fixation element and a second fixation element may each defines a respective lateral extent of the fixation device. The implantable fixation may also includes a first gripping element that is moveable relative to the first fixation element between a first position and a second position, a second gripping element that is moveable relative to the second fixation element between a first position and a second position, and a center portion. The system further includes a delivery device that includes a catheter. The catheter has a proximal end and a distal end and defines at least one lumen that extends between the proximal end and the distal end. The delivery device also includes a shaft that extends through the lumen and is releasably coupled to the center portion. Further, the system includes a first optical coherence tomography (“OCT”) catheter that is configured for cardiovascular imaging and includes a first imaging probe. The first imaging probe includes a first end, a second end, and a first lens assembly disposed at the second end of the first imaging probe. The first imaging probe is configured to transmit light between the first and second ends thereof. In an assembled condition of the fixation system, the first imaging probe extends through the at least one lumen and out from the second end of the shaft such that the first lens assembly is positioned between the first and second lateral extents of the fixation device.

Additionally, the first imaging probe may include a sheath and an optical fiber disposed within the sheath. The optical fiber may be rotatable and translatable within the sheath. Also, the first lens assembly may include a lens and a beam deflector. The first lens assembly may be disposed at an end of the optical fiber. The beam deflector may be configured to deflect the light transmitted through the first imaging probe in a direction radially outwardly from a central axis of the imaging probe. The beam deflector may also be configured to deflect the light at a perpendicular angle relative to the central axis.

Further, the delivery device may include a handle that may be connected to the first end of the shaft. The handle may have an OCT probe interface configured to receive the first imaging probe and direct it into the lumen of the shaft. Also, the delivery device may include a fixation device control element that may extend through the at least one lumen out from the second end of the shaft where the fixation device control element may engage one of the center portion, the first gripping element, and the second gripping element. The fixation control element may be an actuator rod configured to move the fixation elements from a first position to a second position. The fixation control element may be a gripping element line configured to move the first gripping element or second gripping element from a first position to a second position.

Continuing with this example, a first plane and a second plane may each bisect the center portion. The first plane may be orthogonal to the second plane and may intersect the first and second clamps. The second plane may be located equidistant from each of the first and second clamps. The first lens assembly may be positioned adjacent to the center portion such that the first plane intersects the first lens assembly. Alternatively, the first lens assembly may be positioned adjacent to the center portion such that the second plane intersects the first lens assembly. The first and second planes may define four quadrants arranged about the center portion. In a further alternative, the first lens assembly may be positioned adjacent to the center portion and within one of the four quadrants. The first imaging probe may also extend distally along center portion and proximally into a space between the first gripping element and the first fixation element such that the first lens assembly is positioned within the space. The first fixation element may include a crossbar that may extend across at least a portion of an engagement surface thereof. The first imaging probe may extend between the crossbar and the engagement surface, and the first lens assembly may be positioned one of proximal to the crossbar and distal to the crossbar.

Additionally, the first imaging probe may extend distally along the center portion and proximally along a proximal side of the first gripping element. The first imaging probe may be connected to the first side of the first gripping element.

The system may also include a second OCT catheter that has a second imaging probe and a second lens assembly. The second imaging probe may extend from the shaft of the delivery device such that the second lens assembly may be positioned between the first and second lateral extents.

### BRIEF DESCRIPTION OF THE DRAWINGS

The features, aspects, and advantages of the present invention will become better understood regarding the following description, appended claims, and accompanying drawings in which:

FIG. 1 illustrates a partial cutaway view of the left ventricle and left atrium of the heart during ventricular systole;

FIG. 2A illustrates free edges of leaflets of the mitral valve in normal coaptation, and FIG. 2B illustrates the free edges in regurgitative coaptation;

FIGS. 3A-3C illustrate grasping of the leaflets with a fixation device, inversion of the distal elements of the fixation device and removal of the fixation device, respectively;

FIG. 4 illustrates the position of the fixation device in an example of a desired orientation relative to the leaflets;

FIGS. 5A-5B, 6A-6B illustrate exemplary coupling mechanisms for coupling the fixation device to a shaft of a delivery catheter;

FIG. 7 illustrates an exemplary fixation device coupled to a shaft;

FIGS. 8A-8B, 9A-9B, 10A-10B, 11A-11B, and 12-14 illustrate a fixation device in various example positions during introduction and placement of the device within the body to perform a therapeutic procedure;

FIGS. 15A-15C illustrate a covering on the fixation device wherein the device is in various positions;

FIG. 16 illustrates a fixation device including proximal elements and a locking mechanism;

FIG. 17 is a cross-sectional view of the locking mechanism of FIG. 16;

FIGS. 18-19 are cross-sectional views of the locking mechanism in the unlocked and locked positions respectively;

FIGS. 20A and 20B are schematic insertion force diagrams along proximal and distal elements of a first and second fixation device having different lengths, respectively;

FIG. 21A is a schematic diagram of an example of an OCT catheter system;

FIG. 21B is a partial cutaway view of an OCT probe of the OCT catheter system of FIG. 21A;

FIG. 22A illustrates a perspective view of an embodiment of a delivery catheter for a fixation device;

FIG. 22B is a cross-sectional view taken along line B-B of FIG. 22A;

FIG. 23 is a perspective view of an example of a multi-catheter guiding system according to an embodiment of the present disclosure with a delivery catheter shaft of the delivery catheter of FIG. 22A positioned therethrough;

FIG. 24A is a perspective view of an example of an OCT probe in a central arrangement relative to a fixation device having a covering;

FIGS. 24B and 24C are perspective and front views, respectively, of the OCT probe and fixation device arrangement of FIG. 24A with the fixation device shown without the covering;

FIG. 24D is a cross-sectional schematic of the OCT probe and fixation device arrangement of FIG. 24A taken at a 50% leaflet insertion position represented by line D-D of FIG. 24C;

FIG. 24E is a schematic of the OCT probe and fixation device arrangement of FIG. 24A with valve leaflets inserted within the fixation device;

FIG. 24F is a cross-sectional OCT image of the valve leaflet insertion of FIG. 24E taken at the 50% leaflet insertion position;

FIG. 25 is a cross-sectional schematic of the OCT probe in another central arrangement relative to the fixation device taken at a 50% leaflet insertion position;

FIG. 26 is a cross-sectional schematic of first and second OCT probes in a central arrangement relative to the fixation device taken at a 50% leaflet insertion position;

FIG. 27A is a front view of the first and second OCT probes in another central arrangement relative to the fixation device;

FIG. 27B is a cross-sectional schematic of the first and second OCT probes in the central arrangement of FIG. 27A taken at a 50% leaflet insertion position;

FIG. 28 is a cross-sectional schematic of the first and second OCT probes in a further central arrangement taken at a 50% leaflet insertion position;

FIG. 29 is a cross-sectional schematic of the first and second OCT probes in yet another central arrangement taken at a 50% leaflet insertion position;

FIG. 30A is a front view of an example of a fixation device having a central spacer containing an OCT probe and an actuator rod;

FIG. 30B is a cross-sectional view of the central spacer taken along line B-B of FIG. 30A;

FIG. 31A is a perspective view of first and second OCT probes in a clamp arrangement relative to a fixation device having a covering and within respective distal elements of the fixation device;

FIG. 31B is a perspective view of the clamp arrangement of FIG. 31A with the fixation device shown without the covering;

FIG. 31C is a cross-sectional schematic of the first and second OCT probes in the clamp arrangement of FIG. 31A taken at a 50% leaflet insertion position;

FIG. 31D is perspective view of the first OCT probe coupled to a first distal element of the fixation device of FIG. 31A and in a first lens arrangement relative to a crossbar of the first distal element;

FIG. 32 is perspective view of the second OCT probe coupled to the first distal element of the fixation device of FIG. 31A and in a second lens arrangement relative to a crossbar of the first element;

FIG. 33 is perspective view of the first OCT probe coupled to the first distal element of the fixation device of FIG. 31A and in a third lens arrangement relative to a crossbar of the first distal element;

FIG. 34A is a front view of a first and second OCT probe in another clamp arrangement relative to the fixation device and within respective proximal elements of the fixation device;

FIG. 34B is a cross-sectional schematic of the first and second OCT probes in the clamp arrangement of FIG. 34A taken at a 50% leaflet insertion position; and

FIG. 35 illustrates an example of a mitral valve procedure in which an OCT probe remains positioned within a first fixation device while a second fixation device is implanted.

### DETAILED DESCRIPTION

### I. Introduction

Percutaneous edge-to-edge valve repair, whether it be mitral valve or tricuspid valve repair, is typically performed with a fixation device to varying degrees of success under transesophageal echocardiography (TEE) imaging guidance. TEE is a specialized imaging technique that uses ultrasound to obtain detailed images of the heart and its structures. In edge-to-edge valve repair, a TEE probe, also called a transducer, is inserted into an esophagus posterior to and in close proximity to the heart. From there, the TEE probe uses high-frequency sound waves to generate real-time two-dimensional cross-sectional views and three-dimensional en face views of a targeted valve undergoing repair. The surgeon uses these views to guide the fixation device to the targeted valve and to orient the fixation device at a desired orientation relative to a line of coaptation between opposed leaflets.

However, substantial difficulties arise when it comes time to capture the valve leaflets with the fixation device as TEE is suboptimal for assessing the quality of the leaflet tissue being grasped and for assessing the sufficiency of leaflet capture. In this regard, a surgeon will typically observe changes to the movement of the leaflets (e.g., leaflet flail or lack thereof) during the diastole/systole cycle and reduction in backflow into the relevant atrium using color doppler to estimate that one or more of the leaflets have been captured just before releasing the device into the heart. However, this is just an estimation and is an estimation that is heavily reliant on surgeon experience using suboptimal imaging. The suboptimal nature of TEE for assessing leaflet capture is even more pronounced when performing edge-to-edge repair on a tricuspid valve. The tricuspid valve is located in the anterior mediastinum relatively far away from the standard mid-esophageal position of a TEE probe as compared to a mitral valve potentially resulting lower quality images from which to make the assessment.

Thus, a substantial difficulty that remains for percutaneous edge-to-edge valve repair is determining that quality leaflet tissue has been adequately grasped and captured within the implantable fixation device. Specifically, when leaflet insertion into the device is not sufficient on both sides of the device there may be a heightened chance of a leaflet slipping out from one side of the device (Single Leaflet Device Attachment, SLDA), or an increased chance of both leaflets slipping out of both sides of the device (device embolization). Even where leaflet detachment does not occur, insufficient leaflet insertion can lead to inadequate leaflet approximation and residual valve regurgitation resulting in a poor outcome for the patient. Exacerbating this issue is the current inability to adequately assess the quality of the tissue being captured as calcification and other structural issues at the grasping site may reduce the quality of what might otherwise be a sufficient capture or may indicate that installation of a fixation device is altogether contraindicated.

Alternatives to address the current difficulties of leaflet capture have been contemplated but provide only marginal improvements and introduce additional complications. For example, U.S. Pat. No. 8,758,393 describes a suturing device used to suture artificial chordae tendineae to a valve leaflet. The suturing device uses a fiber optic bundle to distinguish between blood and a valve leaflet between a grasper that is used to shuttle a suture needle through the tissue.

In another example, U.S. Pat. No. 7,635,329 describes a valvular fixation device with an embedded sensor to determine the presence or absence of tissue within the device. The described sensor may be in the form of a conductor, a strain gauge, a radiosensor, an optical sensor, an ultrasound sensor, an infrared sensor, an electrical resistance sensor, an intravascular ultrasound sensor, a pressure sensor, or a resonating sensor responsive to magnetic energy.

While the means described in these examples may differ, the information generated by each of such means is generally of a binary form that simply indicates the presence or absence of tissue within a general space and does not necessarily indicate the length of tissue within a device nor does it identify defects, such as defects in tissue quality or other structural issues. Ultimately, the surgeon does not have good visualization and must assess the quality of the grasp and the quality of the tissue itself using the techniques previously described with respect to TEE. Additionally, the means contemplated by the aforementioned examples generally require a large footprint in an already limited space of a percutaneously delivered fixation device and may require electrically active components to be positioned within an electrically active heart, which is typically undesirable.

The following exemplary devices, systems, and methods address the problems and limitations associated with the related art. Although certain descriptions in the following discussion of such exemplary devices, systems, and methods may be in the context of the treatment of mitral valve regurgitation, it should be understood that such discussion of the same is also applicable to the treatment of tricuspid valve regurgitation.

### A. Cardiac Physiology

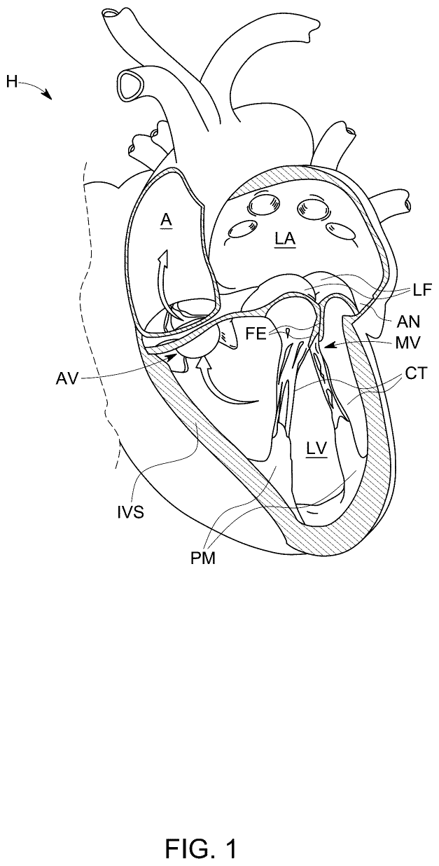

The left ventricle (LV) of a normal heart H in ventricular systole is illustrated in FIG. 1. The left ventricle (LV) is contracting and blood flows outwardly through the aortic valve (AV) in the direction of the arrows. Back flow of blood or “regurgitation” through the mitral valve (MV) is prevented since the mitral valve is configured as a “check valve” which prevents back flow when pressure in the left ventricle is higher than that in the left atrium (LA). The mitral valve (MV) comprises a pair of leaflets having free edges (FE) which meet evenly to close, as illustrated in FIG. 1. The opposite ends of the leaflets (LF) are attached to the surrounding heart structure along an annular region referred to as the annulus (AN). The free edges (FE) of the leaflets (LF) are secured to the lower portions of the left ventricle LV through chordae tendineae (CT) (referred to hereinafter as the chordae) which include a plurality of branching tendons secured over the lower surfaces of each of the valve leaflets (LF). The chordae (CT) in turn, are attached to the papillary muscles (PM) which extend upwardly from the lower portions of the left ventricle and intraventricular septum (IVS).

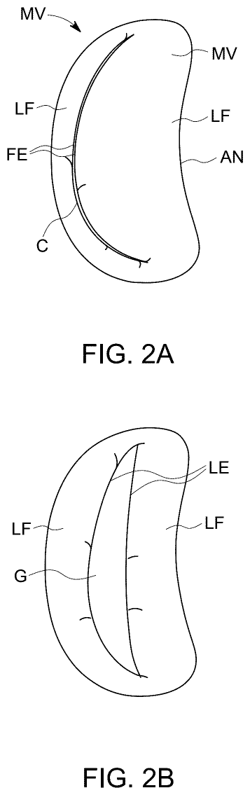

Numerous structural defects in the heart can cause mitral valve regurgitation. Regurgitation occurs when the valve leaflets do not close properly allowing leakage from the ventricle into the atrium. As shown in FIG. 2A, the free edges of the anterior and posterior leaflets (LF) normally meet along a line of coaptation (C). An example of a defect causing regurgitation is shown in FIG. 2B. Here an enlargement of the heart causes the mitral annulus to become enlarged, making it impossible for the free edges (FE) to meet during systole. This results in a gap (G) which allows blood to leak through the valve during ventricular systole. Ruptured or elongated chordae can also cause a valve leaflet to prolapse since inadequate tension is transmitted to the leaflet via the chordae. While the other leaflet maintains a normal profile, the two valve leaflets do not properly meet and leakage from the left ventricle into the left atrium will occur. Such regurgitation can also occur in patients who have suffered ischemic heart disease where the left ventricle does not contract sufficiently to effect proper closure.

### II. General Overview of Valve Fixation Technology

Fixation devices are used for grasping, approximating and fixating tissues such as valve leaflets to treat cardiac valve regurgitation, particularly mitral valve regurgitation and, to a lesser extent, tricuspid valve regurgitation. The fixation devices may also provide features that allow repositioning and removal of the device if so desired, particularly in areas where removal may be hindered by anatomical features such as chordae CT. Such removal would allow the surgeon to reapproach the valve in a new manner if so desired.

Grasping will preferably be atraumatic, providing several benefits. By atraumatic, it means that the devices and methods may be applied to the valve leaflets and then removed without causing any significant clinical impairment of leaflet structure or function. The leaflets and valve continue to function substantially the same as before the fixation devices are applied. Thus, some minor penetration or denting of the leaflets may occur using the devices while still meeting the definition of “atraumatic.” Similarly, during disabling or removal of the fixation device, a small portion of the leaflet(s) may be cut around the edges of the fixation device. Such atraumatic installation, disabling, or removal enables the devices to be applied to a diseased valve and, if desired, removed or repositioned without having negatively affected valve function. In addition, it will be understood that in some cases it may be necessary or desirable to pierce or otherwise permanently affect the leaflets during either grasping, fixing and/or removal. In some cases, grasping and fixation may be accomplished by a single device.

The fixation devices may rely upon the use of an interventional tool that is positioned near a desired treatment site and used to grasp the target tissue. In endovascular applications, the interventional tool is typically an interventional catheter. In surgical applications, the interventional tool is typically an interventional instrument. Fixation of the grasped tissue is accomplished by maintaining grasping with a portion of the interventional tool which is left behind as an implant. The fixation devices are well adapted for the repair of valves, especially cardiac valves such as the mitral valve.

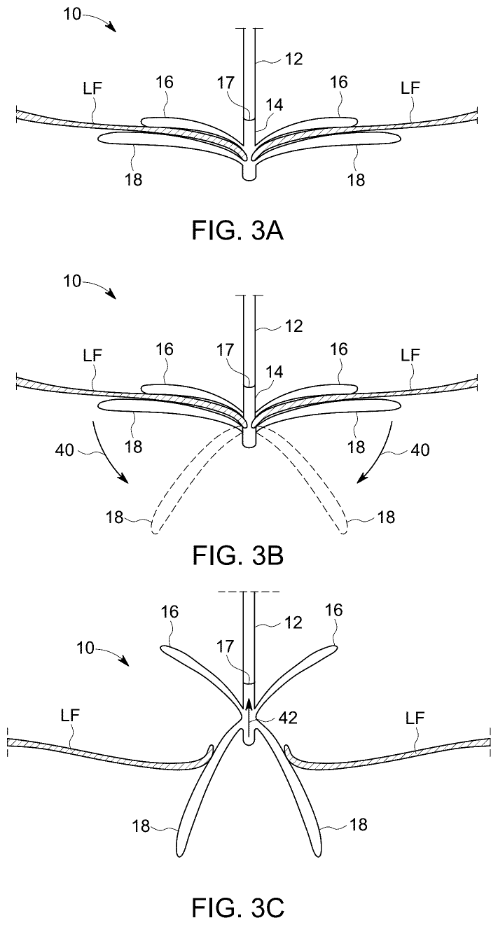

Referring in addition to FIG. 3A, FIG. 3A illustrates an example of an interventional tool 10, having a delivery device, such as a shaft 12, and a fixation device 14, is illustrated having approached the mitral valve MV from the atrial side and grasped the leaflets LF. The mitral valve may be accessed either surgically or by using endovascular techniques, and either by a retrograde approach through the ventricle or by an antegrade approach through the atrium, as described above. For illustration purposes, an antegrade approach is described.

The fixation device 14 is releasably attached to shaft 12 of the interventional tool 10 at its distal end. When describing the devices of the invention herein, “proximal” shall mean the direction toward the end of the device to be manipulated by the user outside the patient's body, and “distal” shall mean the direction toward the working end of the device that is positioned at the treatment site and away from the user. With respect to the mitral valve, proximal shall refer to the atrial or upstream side of the valve leaflets and distal shall refer to the ventricular or downstream side of the valve leaflets.

The fixation device 14 typically comprises proximal elements 16 (or gripping elements) and distal elements 18 (or fixation elements) which protrude radially outward and are positionable on opposite sides of the leaflets LF as shown to capture or retain the leaflets therebetween. A combination of one proximal element 16 and one distal element is referred to herein as a clamp or a clip as such features operate together to clamp, clip, pinch, or otherwise grasp tissue, as explained in further detail below. The proximal elements 16 are preferably comprised of cobalt chromium, nitinol or stainless steel, and the distal elements 18 are preferably comprised of cobalt chromium or stainless steel, however any suitable materials may be used. The fixation device 14 is coupleable to the shaft 12 by a coupling mechanism 17. The coupling mechanism 17 allows the fixation device 14 to detach and be left behind as an implant to hold the leaflets together in the coapted position.

In some situations, it may be desired to reposition or remove the fixation device 14 after the proximal elements 16, distal elements 18, or both have been deployed to capture the leaflets LF. Such repositioning or removal may be desired for a variety of reasons, such as to reapproach the valve in an attempt to achieve better valve function, more optimal positioning of the device 14 on the leaflets, better purchase on the leaflets, to detangle the device 14 from surrounding tissue such as chordae, to exchange the device 14 with one having a different design, or to abort the fixation procedure, to name a few. To facilitate repositioning or removal of the fixation device 14, the distal elements 18 may be released and optionally inverted to a configuration suitable for withdrawal of the device 14 from the valve without tangling or interfering with or damaging the chordae, leaflets or other tissue. According to another embodiment, any of the endovascular methods described herein for disabling or removal of the fixation device may also be used.

Reference is now additionally made to FIG. 3B, which illustrates inversion wherein the distal elements 18 are moveable in the direction of arrows 40 to an inverted position. Likewise, the proximal elements 16 may be raised, if desired. In the inverted position, device 14 may be repositioned to a desired orientation wherein the distal elements may then be reverted to a grasping position against the leaflets as in FIG. 3A. Alternatively, the fixation device 14 may be withdrawn (indicated by arrow 42) from the leaflets as shown in FIG. 3C. Such inversion reduces trauma to the leaflets and minimizes any entanglement of the device with surrounding tissues. Once device 14 has been withdrawn through the valve leaflets, the proximal and distal elements may be moved to a closed position or configuration suitable for removal from the body or for reinsertion through the mitral valve.

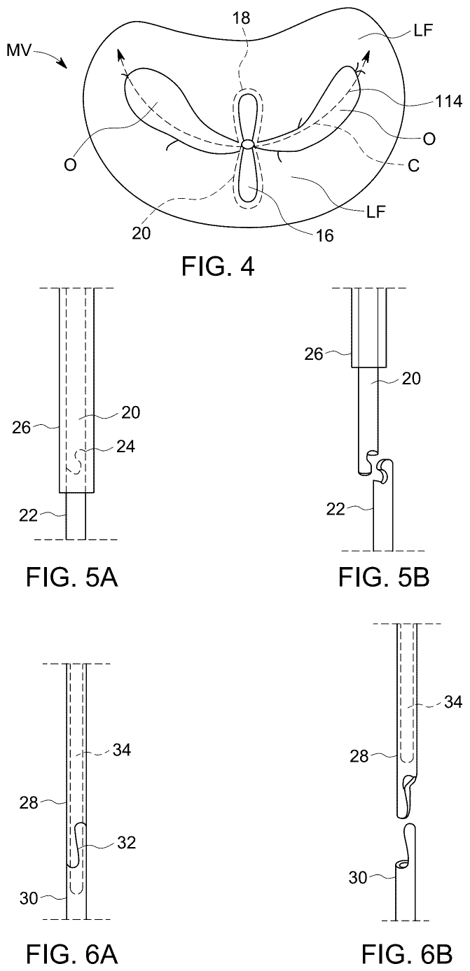

Reference is now additionally made to FIG. 4, which illustrates the position of fixation device 14 in an example of a desired orientation in relation to the leaflets LF. This is a short-axis view of the mitral valve MV from the atrial side, therefore, the proximal elements 16 are shown in solid line and the distal elements 18 are shown in dashed line. The proximal and distal elements 16, 18 are positioned to be substantially perpendicular to the line of coaptation C according to an example of the disclosure. In one example, the device 14 may be moved roughly along the line of coaptation to the location of regurgitation. The leaflets LF are held in place so that during ventricular diastole, as shown in FIG. 4, the leaflets LF remain in position between the elements 16, 18 surrounded by openings or orifices O which result from the diastolic pressure gradient. Advantageously, leaflets LF are coapted such that their proximal or upstream surfaces are facing each other in a vertical orientation, parallel to the direction of blood flow through mitral valve MV. The upstream surfaces may be brought together to be in contact with one another or may be held slightly apart but will preferably be maintained in the vertical orientation in which the upstream surfaces face each other at the point of coaptation according to an example of the disclosure. This simulates the double orifice geometry of a standard surgical bow-tie repair. As discussed further below, the sufficiency of leaflet capture by fixation device 14 may be assessed. If necessary or otherwise desired, one or more of the leaflets may be released and regrasped until an optimal result is produced wherein the leaflets LF are held in place and regurgitation is substantially reduced.

Once the leaflets are coapted in the desired arrangement, the fixation device 14 is then detached from the shaft 12 and left behind as an implant to hold the leaflets together in the coapted position. As described above, in some examples, the fixation device 14 is coupled to the shaft 12 by a coupling mechanism 17. FIGS. 5A-5B, 6A-6B illustrate examples of such coupling mechanisms. FIG. 5A shows an upper shaft 20 and a detachable lower shaft 22 which are interlocked at a joining line or mating surface 24. The mating surface 24 may have any shape or curvature which will allow or facilitate interlocking and later detachment. A snugly fitting outer sheath 26 is positioned over the shafts 20, 22 to cover the mating surface 24 as shown. FIG. 5B illustrates detachment of the lower shaft 22 from the upper shaft 20 according to various examples. In such examples, this may be achieved by retracting the outer sheath 26, so that the mating surface 24 is exposed, which allows the shafts 20, 22 to separate.

Referring now in addition to FIG. 6A which similarly illustrates a tubular upper shaft 28 and a detachable tubular lower shaft 30 which are interlocked at a mating surface 32. Again, the mating surface 32 may have any shape or curvature which will allow or facilitate interlocking and later detachment. In one example, the tubular upper shaft 28 and tubular lower shaft 30 form an outer member having an axial channel. A snugly fitting rod 34 or inner member is inserted through the tubular shafts 28, 30 to bridge the mating surface 32 as shown. FIG. 6B illustrates detachment of the lower shaft 30 from the upper shaft 28. This is achieved by retracting rod 34 to a position above the mating surface 32 which in turn allows the shafts 28, 30 to separate according to an example of the disclosure.

In one example, the mating surface 24 (or mating surface 32) is a sigmoid curve defining a male element and female element on upper shaft 20 (or upper shaft 28) which interlock respectively with corresponding female and male elements on lower shaft 22 (or lower shaft 30). In one example, the lower shaft is coupling mechanism 17 of the fixation device 14. Therefore, the shape of the mating surface selected will preferably provide at least some mating surfaces transverse to the axial axis of the mechanism 19 to facilitate application of compressive and tensile forces through the coupling mechanism 17 to the fixation device 14 yet causing minimal interference when the fixation device 14 is to be released from the upper shaft. It will be appreciated that these coupling mechanisms are exemplary, and other coupling mechanisms could also be used.

### A. Exemplary Fixation Device

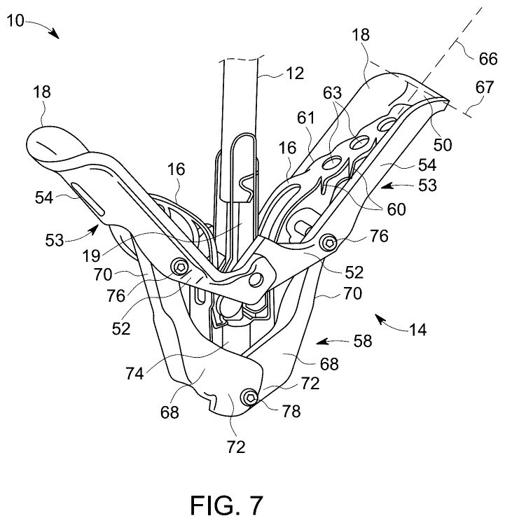

Referring also to FIG. 7, which illustrates an exemplary fixation device 14. Here, the fixation device 14 is shown coupled to a shaft 12 to form an interventional tool 10. The fixation device 14 includes a coupling member 19 and a pair of opposed distal elements 18. The distal elements 18 comprise elongate arms 53, each arm having a proximal end 52 rotatably connected to the coupling member 19 and a free end 54. The free ends 54 have a rounded shape to minimize interference with and trauma to surrounding tissue structures. Preferably, each free end 54 defines a curvature about two axes, one being an axis 66 perpendicular to longitudinal axis of arms 53. Thus, the engagement surfaces 50 have a cupped or concave shape to the surface area in contact with tissue and to assist in grasping and holding the valve leaflets. This further allows arms 53 to nest around the shaft 12 in the closed position to minimize the profile of the device. Preferably, arms 53 are at least partially cupped or curved inwardly about their longitudinal axes 66. Also, preferably, each free end 54 defines a curvature about an axis 67 perpendicular to axis 66 or the longitudinal axis of arms 53. This curvature is a reverse curvature along the most distal portion of the free end 54. Likewise, the longitudinal edges of the free ends 54 may flare outwardly in some examples. Both the reverse curvature and flaring minimize trauma to the tissue engaged therewith.

To be suitable for mitral valve repair, the transverse width across engagement surfaces 50 (which determines the width of tissue engaged) may be at least about 2 mm, usually 3-10 mm, and preferably about 4-6 mm according to various non-limiting examples. In some situations, a wider engagement is desired wherein the engagement surfaces 50 are larger, for example about 2 cm, or multiple fixation devices are used adjacent to each other. Arms 53 and engagement surfaces 50 are configured to engage a length of tissue of about 4-10 mm, and preferably about 6-8 mm along the longitudinal axis of arms 53 in some examples. In various examples, arms 53 further include a plurality of openings to enhance grip and to promote tissue ingrowth following implantation.

The valve leaflets are grasped between the distal elements 18 and proximal elements 16. The proximal elements 16 may be flexible, resilient, and cantilevered from coupling member 19 in some examples. The proximal elements are preferably resiliently biased toward the distal elements. In one example, each proximal element 16 is shaped and positioned to be at least partially recessed within the concavity of the distal element 18 when no tissue is present. According to one example, when the fixation device 14 is in the open position, the proximal elements 16 are shaped such that each proximal element 16 is separated from the engagement surface 50 near the proximal end 52 of arm 53 and slopes toward the engagement surface 50 near the free end 54 with the free end of the proximal element contacting engagement surface 50, as illustrated in FIG. 7. This shape of the proximal elements 16 accommodates valve leaflets or other tissues of varying thicknesses.

Proximal elements 16 include a plurality of openings 63 and scalloped side edges 61 to increase grip on tissue in various examples. Proximal elements 16 optionally include frictional accessories, frictional features, or grip-enhancing elements to assist in grasping and/or holding the leaflets. The frictional accessories may comprise tines or barbs, for example, 60 having tapering pointed tips extending toward engagement surfaces 50. Any suitable frictional accessories may be used, such as prongs, windings, bands, barbs, grooves, channels, bumps, surface roughening, sintering, high-friction pads, coverings, coatings, or a combination of these. Optionally, magnets may be present in the proximal and/or distal elements. It may be appreciated that the mating surfaces may be made from or may include material of opposite magnetic charge to cause attraction by magnetic force. For example, the proximal elements and distal elements may each include magnetic material of opposite charge so that tissue is held under constant compression between the proximal and distal elements to facilitate faster healing and ingrowth of tissue. Also, the magnetic force may be used to draw the proximal elements 16 toward the distal elements 18, in addition to or alternatively to biasing of the proximal elements toward the distal elements. This may assist in deployment of the proximal elements 16. In another example, the distal elements 18 each include magnetic material of opposite charge so that tissue positioned between the distal elements 18 is held therebetween by magnetic force.

Proximal elements 16 may be covered with a fabric or other flexible material as described below to enhance grip and tissue ingrowth following implantation. Preferably, when fabrics or coverings are used in combination with barbs or other frictional features, such features will protrude through such fabric or other covering so as to contact any tissue engaged by proximal elements 16.

In various examples, proximal elements 16 may be formed from metallic sheet of a spring-like material using a stamping operation which creates openings 63, scalloped edges 61 and barbs 60. Alternatively, proximal elements 16 could be comprised of a spring-like material or molded from a biocompatible polymer. Some types of frictional accessories may permanently alter or cause some trauma to the tissue engaged thereby, whereas other frictional accessories will be atraumatic and will not injure or otherwise affect the tissue in a clinically significant way. For example, in the case of barbs 60, it has been demonstrated that following engagement of mitral valve leaflets by fixation device 14, should the device later be removed during the procedure barbs 60 leave no significant permanent scarring or other impairment of the leaflet tissue and are thus considered atraumatic.

The fixation device 14 also includes an actuation mechanism 58 in various examples. In some examples, the actuation mechanism 58 comprises two link members or legs 68, each leg 68 having a first end 70 which is rotatably joined with one of the distal elements 18 at a riveted joint 76 and a second end 72 which is rotatably joined with a stud 74. Legs 68 are preferably comprised of a rigid or semi-rigid metal or polymer such as Elgiloy®, cobalt chromium or stainless steel, however any suitable material may be used. While in the illustrated device both legs 68 are pinned to stud 74 by a single rivet 78, it may be appreciated, however, that each leg 68 may be individually attached to the stud 74 by a separate rivet or pin. The stud 74 is joinable with an actuator rod 64 (not shown) which extends through the shaft 12 and is axially extendable and retractable to move the stud 74 and therefore the legs 68 which rotate the distal elements 18 between closed, open and inverted positions. Likewise, immobilization of stud 74 holds the legs 68 in place and therefore holds the distal elements 18 in a desired position. The stud 74 may also be locked in place by a locking feature which will be further described in later sections.

In some examples, there may be some mobility or flexibility in distal elements 18 and/or proximal elements 16 of the fixation device 14 in the closed position to enable these elements to move or flex with the opening or closing of the valve leaflets. This provides shock absorption and thereby reduces force on the leaflets and minimizes the possibility for tearing or other trauma to the leaflets. Such mobility or flexibility may be provided by using a flexible, resilient metal or polymer of appropriate thickness to construct the distal elements 18. Also, in some examples, the locking mechanism of the fixation device (described below) may be constructed of flexible materials to allow some slight movement of the proximal and distal elements even when locked. Further, in some examples, the distal elements 18 can be connected to the coupling mechanism 19 or to actuation mechanism 58 by a mechanism that biases the distal element to the closed position (inwardly) but permits the arms to open slightly in response to forces exerted by the leaflets. For example, rather than being pinned at a single point, these components may be pinned through a slot that allows a small amount of translation of the pin in response to forces against the arms. A spring may be used to bias the pinned component toward one end of the slot.

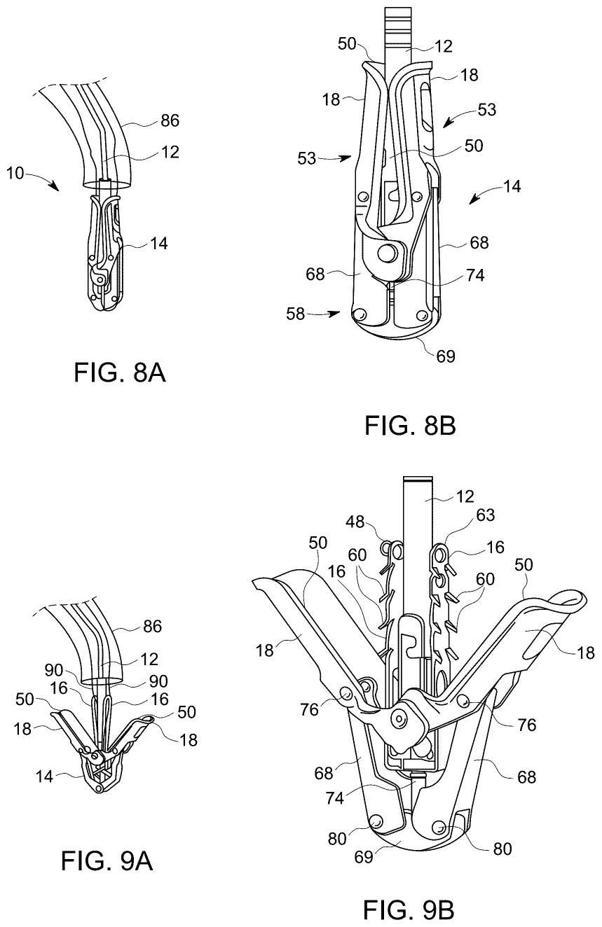

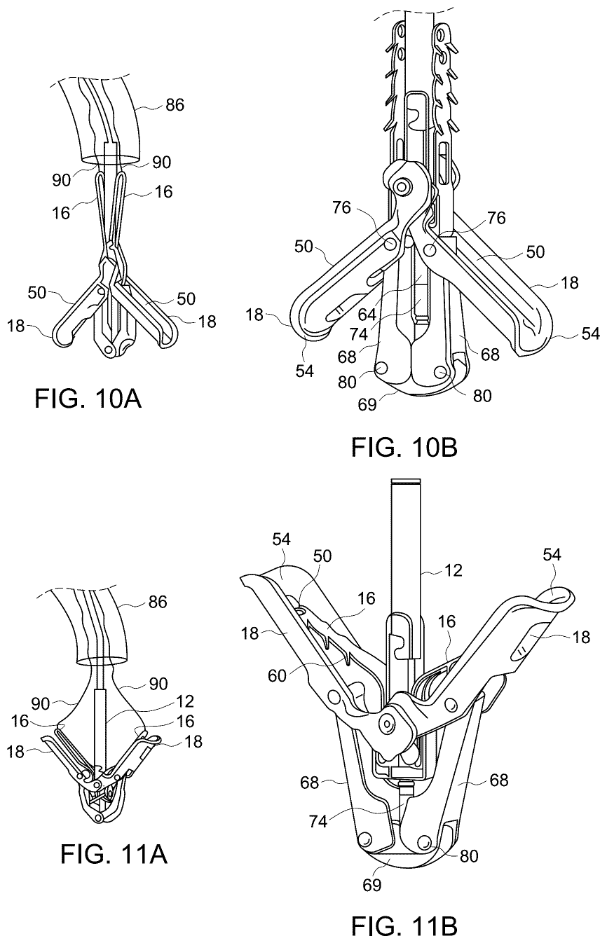

Referring in addition to FIGS. 8A-8B, 9A-9B, 10A-10B, 11A-11B, and 12-14, which illustrate various possible positions of the fixation device 14 of FIG. 7 during introduction and placement of the device 14 within the body to perform a therapeutic procedure. FIG. 8A illustrates an example of an interventional tool 10 delivered through a catheter 86. It should be appreciated that the interventional tool 10 may take the form of a catheter, and likewise, the catheter 86 may take the form of a guide catheter or sheath. However, in this example the terms interventional tool 10 and catheter 86 will be used. Interventional tool 10 comprises a fixation device 14 coupled to a shaft 12 and the fixation device 14 is shown in the closed position. FIG. 8B illustrates a device similar to the device of FIG. 8A in a larger view. In the closed position, the opposed pair of distal elements 18 are positioned so that the engagement surfaces 50 face each other. Each distal element 18 comprises an elongate arm 53 having a cupped or concave shape so that together the arms 53 surround the shaft 12 and optionally contact each other on opposite sides of the shaft. This provides a low profile for the fixation device 14 which is readily passable through catheter 86 and through relevant anatomical structures, such as the mitral valve. In addition, FIG. 8B further shows an example of an actuation mechanism 58. The actuation mechanism 58 comprises two legs 68 which are each movably coupled to a base 69. In one example, base 69 is joined with an actuator rod 64 (see FIG. 10B) which extends through shaft 12 and is used to manipulate the fixation device 14. The actuator rod 64 may attach directly to the actuation mechanism 58, particularly the base 69. However, the actuator rod 64 may alternatively attach to a stud 74 which in turn is attached to the base 69. The stud 74 may be threaded so that the actuator rod 64 attaches to the stud 74 by a screw-type action. However, the rod 64 and stud 74 may be joined by any mechanism which is releasable to allow the fixation device 14 to be detached from shaft 12.

Referring in addition to FIGS. 9A-9B, which illustrate the fixation device 14 in the open position. In the open position, the distal elements 18 are rotated so that the engagement surfaces 50 face a first direction according to an example of the disclosure. For example, distal advancement of the stud 74 relative to coupling member 19 by action of the actuator rod 64 applies force to the distal elements 18 which begin to rotate around joints 76 due to freedom of movement in this direction. Such rotation and movement of the distal elements 18 radially outward causes rotation of the legs 68 about joints 80 so that the legs 68 are directed slightly outwards. Stud 74 may be advanced to any desired distance correlating to a desired separation of the distal elements 18. In the open position, engagement surfaces 50 are disposed at an acute angle relative to shaft 12 and are preferably at an angle of between 90 and 180 degrees relative to each other. In various examples of the open position, the free ends 54 of arms 53 may have a span therebetween of about 10-20 mm, usually about 12-18 mm, and preferably about 14-16 mm.

Proximal elements 16 are biased outwardly toward arms 53 in one example. The proximal elements 16 may be moved inwardly toward the shaft 12 and held against the shaft 12 with the aid of proximal element lines 90 which can be in the form of sutures, wires, nitinol wire, rods, cables, polymeric lines, or other suitable structures in some examples. The proximal element lines 90 may be connected with the proximal elements 16 by threading the lines 90 in a variety of ways which allow proximal elements 16 to be collectively and/or independently moved toward or away from a respective distal element 18 through the tensioning or de-tensioning of line 90. When the proximal elements 16 have a loop shape, as shown in the example of FIG. 9A, the line 90 may pass through the loop and double back. When the proximal elements 16 have an elongate solid shape, as shown in the example of FIG. 9B, the line 90 may pass through one or more of the openings 63 in the element 16. Further, a line loop 48 may be present on a proximal element 16, also illustrated in FIG. 9B, through which a proximal element line 90 may pass and double back. Such a line loop 48 may be useful to reduce friction on proximal element line 90 or when the proximal elements 16 are solid or devoid of other loops or openings through which the proximal element lines 90 may attach. A proximal element line 90 may attach to the proximal elements 16 by detachable means which would allow a single line 90 to be attached to a proximal element 16 without doubling back and would allow the single line 90 to be detached directly from the proximal element 16 when desired. Examples of such detachable means include hooks, snares, clips or breakable couplings, to name a few.

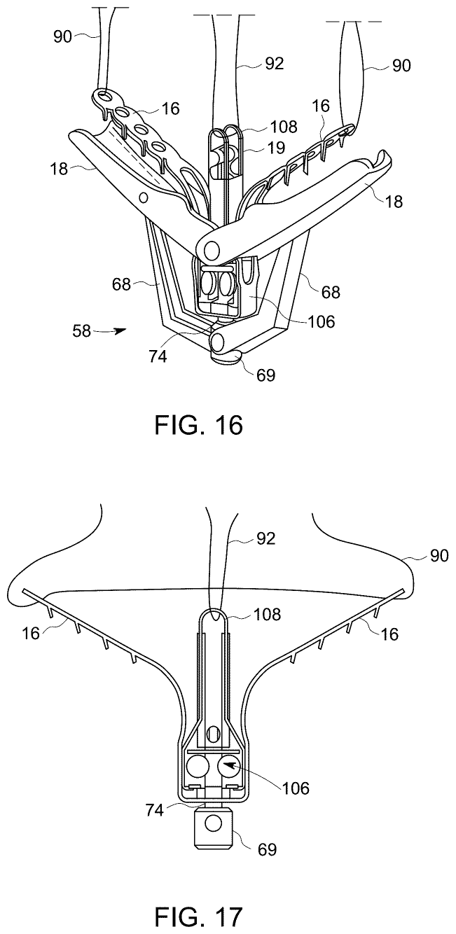

By applying sufficient tension to the proximal element line 90, the detachable means may be detached from the proximal element 16 such as by breakage of the coupling. Other mechanisms for detachment may also be used. Similarly, lock line 92 (FIG. 16) may be attached and detached from a locking mechanism by similar detachable means.

In the open position, the fixation device 14 can engage the tissue which is to be approximated or treated. The device illustrated in FIGS. 7-9B is adapted for repair of the mitral valve using an antegrade approach from the left atrium according to one example of the disclosure. Interventional tool 10 is advanced through the mitral valve from the left atrium to the left ventricle. The distal elements 18 are oriented to be perpendicular to the line of coaptation and then positioned so that the engagement surfaces 50 contact the ventricular surface of the valve leaflets, thereby grasping the leaflets. The proximal elements 16 remain on the atrial side of the valve leaflets so that the leaflets lie between the proximal and distal elements. Proximal elements 16 have frictional accessories, such as barbs 60 which are directed toward the distal elements 18. However, in one example, neither the proximal elements 16 nor the barbs 60 contact the leaflets at this time.

The interventional tool 10 may be repeatedly manipulated to reposition the fixation device 14 so that the leaflets are properly contacted or grasped at a desired location. Repositioning is achieved with the fixation device in the open position. In some instances, regurgitation may also be checked while device 14 is in the open position. If regurgitation is not satisfactorily reduced, the device may be repositioned, and regurgitation checked again until the desired results are achieved.

It may also be desired to invert the fixation device 14 to aid in repositioning or removal of the fixation device 14. Referring also to FIGS. 10A-10B, FIGS. 10A-10B illustrate the fixation device 14 in an example of the inverted position. By further advancement of stud 74 relative to coupling member 19, the distal elements 18 are further rotated so that the engagement surfaces 50 face outwardly and free ends 54 point distally, with each arm 53 forming an obtuse angle relative to shaft 12.

In various examples, the angle between arms 53 is preferably in the range of about 270 to 360 degrees when in the inverted position. Further advancement of the stud 74 further rotates the distal elements 18 around joints 76. This rotation and movement of the distal elements 18 radially outward causes rotation of the legs 68 about joints 80 so that the legs 68 are returned toward their initial position, generally parallel to each other. Stud 74 may be advanced to any desired distance correlating to a desired inversion of the distal elements 18. In some preferred examples, in the fully inverted position, the span between free ends 54 is no more than about 20 mm, usually less than about 16 mm, and most preferably about 12-14 mm. In this illustration, the proximal elements 16 remain positioned against the shaft 12 by exerting tension on the proximal element lines 90. Thus, a relatively large space may be created between the elements 16, 18 for repositioning. In addition, the inverted position allows withdrawal of the fixation device 14 through the valve while minimizing trauma to the leaflets. Engagement surfaces 50 provide an atraumatic surface for deflecting tissue as the fixation device is retracted proximally. Barbs 60 are angled slightly in the distal direction (away from the free ends of the proximal elements 16), reducing the risk that the barbs will catch on or lacerate tissue as the fixation device is withdrawn.

Once the fixation device 14 has been positioned in a desired location against the valve leaflets, the leaflets may then be captured between the proximal elements 16 and the distal elements 18. Referring now in addition to FIGS. 11A-11B, which illustrate the fixation device 14 in an example of such a position. In this example, the proximal elements 16 are lowered toward the engagement surfaces 50 so that the leaflets are held therebetween. In FIG. 11B, the proximal elements 16 are shown to include barbs 60 which may be used to provide atraumatic gripping of the leaflets. Alternatively, larger, more sharply pointed barbs or other penetration structures may be used to pierce the leaflets to more actively assist in holding them in place. This position is similar to the open position of FIGS. 9A-9B, however the proximal elements 16 are now lowered toward arms 53 by releasing tension on proximal element lines 90 to compress the leaflet tissue therebetween. At any time, the proximal elements 16 may be raised and the distal elements 18 adjusted or inverted to reposition the fixation device 14 if regurgitation is not sufficiently reduced.

After the leaflets have been captured between the proximal and distal elements 16, 18 in a desired arrangement, the distal elements 18 may be locked to hold the leaflets in this position or the fixation device 14 may be returned to or toward a closed position according to some examples of the disclosure. Such locking examples are described in a later section. Referring now in addition to FIG. 12, FIG. 12 illustrates the fixation device 14 in the closed position wherein the leaflets (not shown) are captured and coapted. In one example, this is achieved by retraction of the stud 74 proximally relative to coupling member 19 so that the legs 68 of the actuation mechanism 58 apply an upwards force to the distal elements 18 which in turn rotate the distal elements 18 so that the engagement surfaces 50 again face one another. The released proximal elements 16, which are biased outwardly toward distal elements 18, are concurrently urged inwardly by the distal elements 18 in some examples. In various examples, the fixation device 14 may then be locked to hold the leaflets in this closed position as described below.

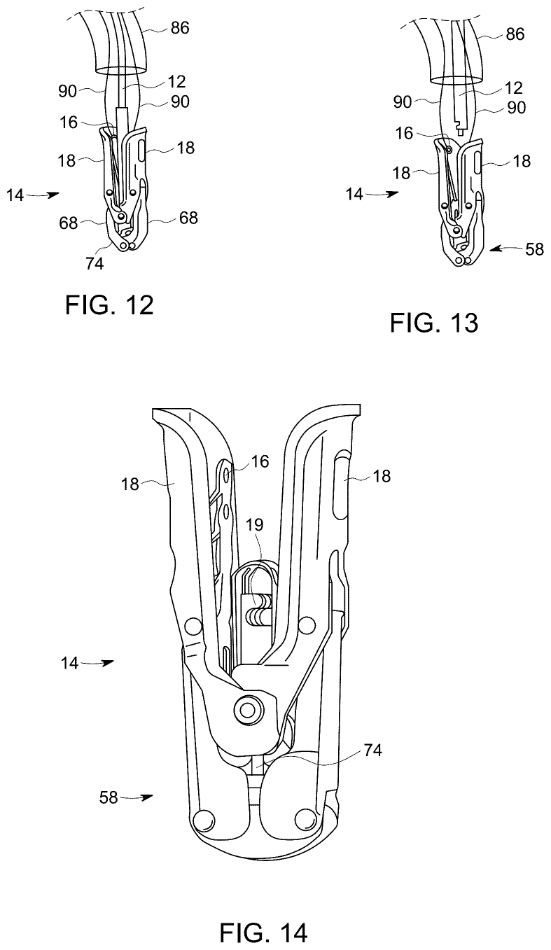

Referring now in addition to FIG. 13, the fixation device 14 may then be released from shaft 12. As mentioned, in various examples, the fixation device 14 is releasably coupleable to the shaft 12 by coupling member 19. FIG. 13 illustrates the coupling structure, a portion of the shaft 12 to which the coupling member 19 of the fixation device 14 attaches according to one example. As shown, in one example, the proximal element lines 90 may remain attached to the proximal elements 16 following detachment from shaft 12 to function as a tether to keep the fixation device 14 connected with the catheter 86. Optionally, a separate tether coupled between shaft 12 and fixation device 14 may be used expressly for this purpose while the proximal element lines 90 are removed. In any case, the repair of the leaflets or tissue may have been observed by non-invasive visualization techniques, such as echocardiography, to ensure the desired outcome. Then if the repair was not as desired, the fixation device 14 could be retrieved with the use of the tether or proximal element lines 90 so as to reconnect coupling member 19 with shaft 12.

In various examples, the proximal element lines 90 may be elongated flexible threads, wire, cable, sutures or lines extending through shaft 12, looped through proximal elements 16, and extending back through shaft 12 to its proximal end. When detachment is desired, one end of each line may be released at the proximal end of shaft 12 and the other end pulled to draw the free end of the line distally through shaft 12 and through proximal element 16 thereby releasing the fixation device.

Referring now in addition to FIG. 14, which illustrates a released fixation device 14 in an example of a closed position. As shown, coupling member 19 remains separated from shaft 12 of the interventional tool 10 and the proximal elements 16 are deployed so that tissue (not shown) may reside between the proximal elements 16 and distal elements 18.

Instead of using a push-to-open, pull-to-close mechanism for opening and closing distal elements 18, a pull-to-open, push-to-close mechanism may also be used in various examples. For example, distal elements 18 may be coupled at their proximal ends to stud 74 rather than to coupling member 19, and legs 68 may be coupled at their proximal ends to coupling member 19 rather than to stud 74. In this example, when stud 74 is pushed distally relative to coupling member 19, distal elements 18 would close, while pulling on stud 74 proximally toward coupling member 19 would open distal elements 18.

### B. Covering on Fixation Device

The fixation device 14 may optionally include a covering. The covering may assist in grasping the tissue and may later provide a surface for tissue ingrowth. Ingrowth of the surrounding tissues, such as the valve leaflets, provides stability to the device 14 as it is further anchored in place and may cover the device with native tissue, thus reducing the possibility of immunologic reactions. In various examples, the covering may be comprised of any biocompatible material, such as polyethylene terephthalate (PET), polyester, cotton, polyurethane, expanded polytetrafluoroethylene (ePTFE), silicone, or various polymers or fibers and have any suitable form, such as a fabric, mesh, textured weave, felt, looped or porous structure. Generally, in some examples, the covering has a low profile so as not to interfere with delivery through an introducer sheath or with grasping and coapting of leaflets or tissue.

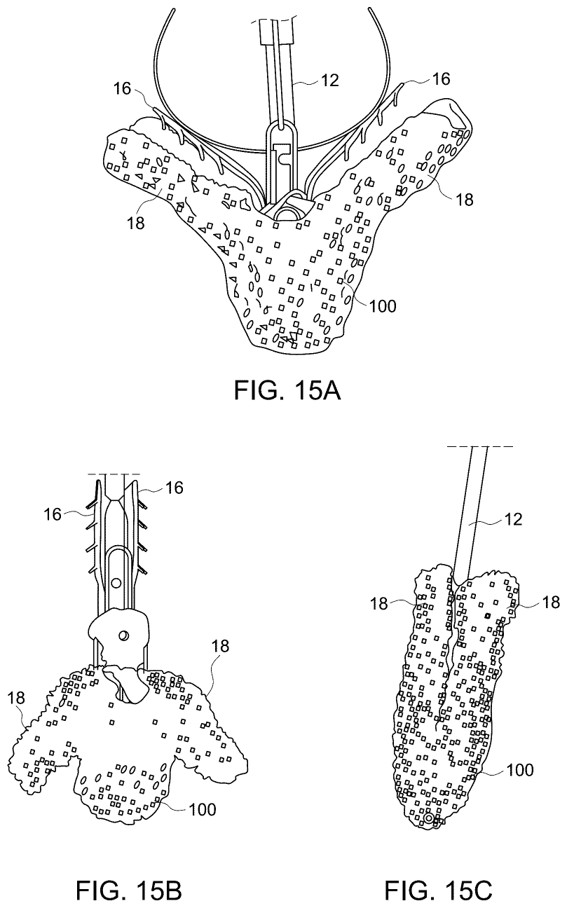

Referring now in addition to FIGS. 15A-15C, which illustrate an example covering 100 on the fixation device 14 while the device 14 is in various positions. Additional description regarding such coverings may be found in PCT Publication No. WO 2004/103162, the disclosure of which is incorporated herein by reference in its entirety.

### C. Locking Mechanism

As mentioned above, the fixation device 14 optionally includes a locking mechanism for locking the device 14 in a particular position, such as an open, closed or inverted position or any position therebetween. It should be appreciated that the locking mechanism includes an unlocking mechanism which allows the device to be either locked or unlocked. Various locking mechanisms can be used with the fixation device 14, such as those described in PCT Publication No. WO 2004/103162, which is incorporated herein by reference in its entirety. Referring now in addition to FIGS. 16-19 that illustrate an exemplary locking mechanism 106. Referring to the example of FIG. 16, locking mechanism 106 is disposed between coupling member 19 and the base 69 of the actuation mechanism 58. Base 69 is fixedly attached to stud 74 which extends through the locking mechanism 106. The stud 74 is releasably attached to the actuator rod 64 which passes through the coupling member 19 and the shaft 12 of the interventional tool 10. Base 69 is also connected to the legs 68 of the actuation mechanism 58 which are in turn connected to the distal elements 18.

FIG. 16 also illustrates the proximal elements 16, which straddle the locking mechanism and join beneath locking mechanism 106 according to this example. The proximal elements 16 are shown supported by proximal element lines 90. In this example, the proximal elements 16 are raised and lowered by manipulation of the proximal element lines 90. In addition, lock lines 92 are shown connected with a release harness 108 of the locking mechanism 106. Lock lines 92 are used to lock and unlock the locking mechanism 106 as will be described below. The proximal element lines 90 and lock lines 92 may be comprised of any suitable material, typically wire, nitinol wire, cable, suture or thread, to name a few. In addition, the proximal element lines 90 and/or lock lines 92 may include a coating, such as parylene, in some examples. Parylene is a vapor deposited pinhole free protective film which is conformal and biocompatible. It is inert and protects against moisture, chemicals, and electrical charge.

Referring in addition to FIG. 17, FIG. 17 provides a front view of the locking mechanism 106 of FIG. 16. However, in this example the proximal elements 16 are supported by a single proximal element line 90 which is through both proximal elements 16. In this arrangement both elements 16 are raised and lowered simultaneously by action of a single proximal element line 90. Whether the proximal elements 16 are manipulated individually by separate proximal element lines 90 or jointly by a single proximal element line 90, the proximal element lines 90 may extend directly through openings in the proximal elements and/or through a layer or portion of a covering 100 on the proximal elements, or through a suture loop above or below a covering 100.

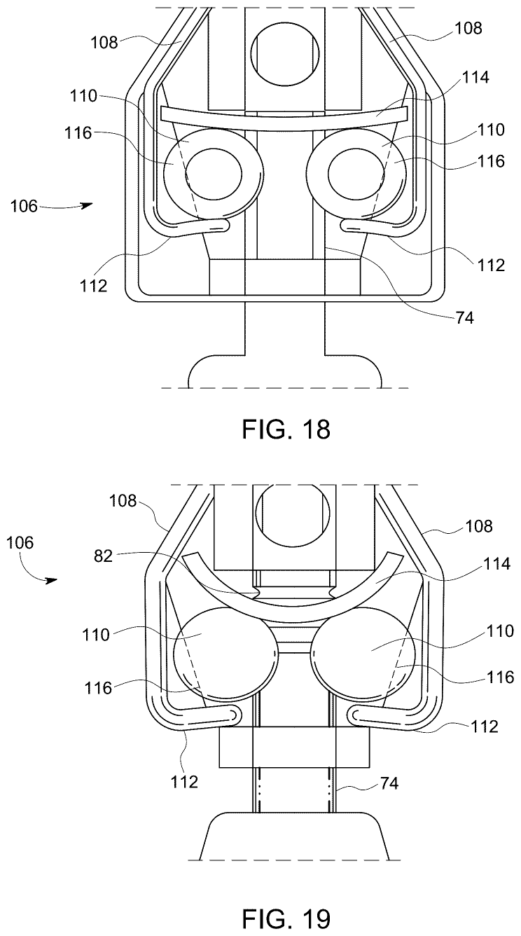

Referring now in addition to FIGS. 18-19, which illustrate locking mechanism 106 showing the locking mechanism 106 in the unlocked and locked positions respectively. Referring to FIG. 18, locking mechanism 106 includes one or more wedging elements, such as rolling elements, in various examples. In this example, the rolling elements comprise a pair of barbells 110 disposed on opposite sides of the stud 74, each barbell having a pair of generally cylindrical caps and a shaft therebetween. The barbells 110 and the stud 74 are preferably comprised of cobalt chromium or stainless steel, however any suitable material may be used. In one example, the barbells 110 are manipulated by hooked ends 112 of the release harness 108. When an upwards force is applied to the harness 108 by the lock line 92 (illustrated in FIG. 16), the hooked ends 112 raise the barbells 110 against a spring 114, as shown in FIG. 18. In one example, this draws the barbells 110 up along a sidewall or sloping surface 116 which unwedges the barbells 110 from against the stud 74. In this position, the stud 74 is free to move. Thus, when the lock line 92 raises or lifts the harness 108, the locking mechanism 106 is in an unlocked position wherein the stud 74 is free to move the actuation mechanism 58 and therefore the distal elements 18 to any desired position. In various examples, release of the harness 108 by the lock line 92 transitions the locking mechanism 106 to a locked position, illustrated in FIG. 19. By releasing the upwards force on the barbells 110 by the hooked ends 112, the spring 114 forces the barbells 110 downwards and wedges the barbells 110 between the sloping surface 116 and the stud 74. This restricts motion of the stud 74, which in turn locks the actuation mechanism 58 and therefore distal elements 18 in place. In addition, in one example, stud 74 may include one or more grooves 82 or indentations which receive the barbells 110. This may provide more rapid and positive locking by causing the barbells 110 to settle in a definite position, increase the stability of the locking feature by further preventing movement of the barbells 110, as well as providing a tangible indication to the user that the barbell has reached a locking position. In addition, the grooves 82 may be used to indicate the relative position of the distal elements 18, particularly the distance between the distal elements 18. For example, each groove 82 may be positioned to correspond with a 0.5 or 1.0 mm decrease in distance between the distal elements 18. As the stud 74 is moved, the barbells 110 will contact the grooves 82; by counting the number of grooves 82 that are felt as the stud 74 is moved, the user can determine the distance between the distal elements 18 and can provide the desired degree of coaptation based upon leaflet thickness, geometry, spacing, blood flow dynamics and other factors. Thus, grooves 82 may provide tactile feedback to the user in various examples of the disclosure.

The locking mechanism 106 allows the fixation device 14 to remain in an unlocked position when attached to the interventional tool 10 during grasping and repositioning and then maintain a locked position when left behind as an implant. It may be appreciated, however, that the locking mechanism 106 may be repeatedly locked and unlocked throughout the placement of the fixation device 14 if desired. Once the final placement is determined, the lock line 92 and proximal element lines 90 are removed and the fixation device is left behind in various examples of the disclosure.

### III. Fixation System With Improved Leaflet Capture Assessment

### A. Leaflet Security

As previously described, opposed valve leaflets may be captured between respective proximal and distal elements 16, 18 to improve leaflet coaptation and reduce valve regurgitation. However, it generally is not sufficient to just capture valve tissue between proximal and distal elements 16, 18. Rather, the valve tissue must be sufficiently secured to realize the benefits of fixation device 14 and to reduce or eliminate the probability of SLDA and/or implant embolization. Leaflet security is generally dictated by leaflet quality and the significance of the pinch force applied to the tissue by fixation device 14. Factors that may affect leaflet quality include leaflet thickness and the presence, amount, and/or distribution of calcification, for example. Leaflets that are too thin may not hold up under the forces applied by the fixation device or may not be able to be sufficiently pinched by the fixation device 12. Calcification can interfere with a fixation device's ability to grasp the tissue and can damage the tissue when grasped.

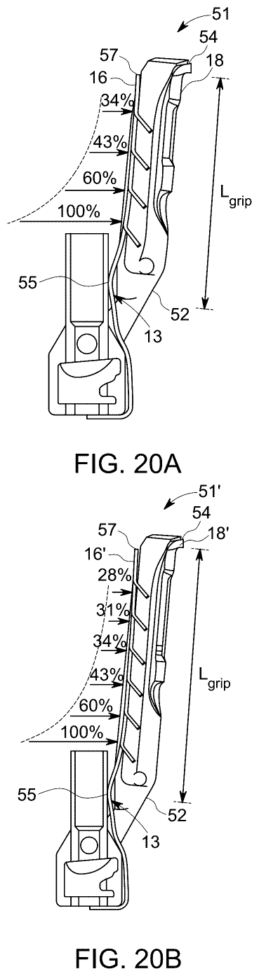

Finite element analysis has been performed to quantify the pinch force within corresponding proximal and distal elements 16, 18 to determine how deep a leaflet should be inserted between proximal element 16 and distal element 18 to ensure leaflet security. The force distribution resulting from this analysis for a first clamp 51 (or first clip 51) comprised of proximal element 16 and distal element 18 is shown in FIG. 20A and for a second, longer clamp 51′ (or second clip 51′) comprised of proximal element 16′ and distal element 18′ is shown in FIG. 20B. As shown, regardless of the length of the clamp 51, 51′, the majority of the generated pinch force is applied to the tissue when approximately more than 50% of a maximum leaflet insertion depth is achieved. Thus, leaflet security is ensured, and leaflet capture is sufficient when a leaflet is inserted between proximal and distal elements 16, 16′, 18, 18′ to a depth of more than 50% of a maximum insertion depth. The maximum insertion depth L_{grip}, as depicted in FIGS. 20A and 20B, is the maximum length along which a leaflet can be captured between the proximal and distal elements 16, 16′, 18, 18′. The maximum insertion depth L_{grip} may be measured between a crotch 13 (taking into account any fabric covering on fixation device 14) formed between distal ends 52, 55 of the proximal and distal elements 16, 16′, 18, 18′ and free ends 54, 57 thereof. Crotch 13 defines a closed end of clamps 51 and 51′. Free ends 54 and 57 define an open end of clamps 51 and 51′. Although proximal and distal elements 16, 16′, 18, 18′ typically capture tissue when each distal element 18, 18′ is at about 60 degrees relative to a longitudinal axis defined by shaft 12 (120 degrees relative each other) and are then moved to a final, closed position of 5 to 15 degrees (10 to 30 degrees relative each other), it should be noted that the pinch force generally does not change with the angle of the clamp 51, 51′ up to about the initial capture angle of 60 degrees. Thus, the pinch force will be approximately the same throughout the range of motion from initial capture to the final, closed position.

While leaflet security is an important aspect to positive outcomes in edge-to-edge repair, up to this point, surgeons have not had sufficient tools to assess leaflet security. TEE is usually relied upon to determine that leaflet capture has been achieved but such assessment is typically just an estimate based on a surgeon's experience of observing leaflet movement characteristics in captured and uncaptured states and a reduction in retrograde flow. TEE does not afford surgeons the ability to accurately and directly determine leaflet security and, in particular, the factors that help ensure leaflet security.

The following describes fixation systems with improved leaflet capture assessment. Such systems generally include a fixation device 14, an Optical Coherence Tomography (“OCT”) catheter system 200, a delivery device 300, and a guide catheter assembly 400 according to various examples of the disclosure.

### B. OCT Catheter System

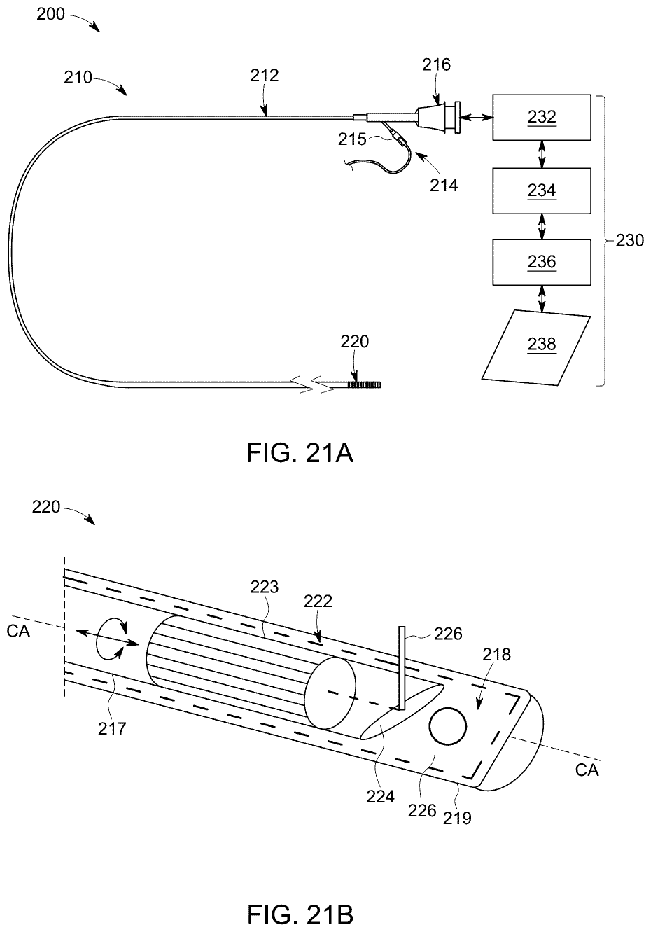

Referring now in addition to FIGS. 21A and 21B, which depict the OCT catheter system 200. In one example, the OCT catheter system 200 generally includes an OCT catheter 210 and an OCT subsystem 230.

OCT catheter or imaging catheter 210 is configured for cardiovascular imaging. OCT catheter 210 may include an imaging probe 212. OCT catheter 210 may also include a flush feature 215. OCT catheter may also include a connector 216. Imaging probe 212 extends from connector 216. Imagine probe 212 includes an optical fiber 217 and a transparent sheath 219 encapsulating optical fiber 217. Optical fiber 217 is configured to transmit light between proximal and distal ends thereof. Optical fiber 217 may be made from a glass or polymer material that is preferably bendable or flexible. Optical fiber 217 is positioned within a lumen 218 of sheath 219 and may be rotatable about and translatable along a central axis CA of imaging probe 212 and relative to sheath 219, as illustrated by the directional arrows shown in FIG. 21B. Alternatively, optical fiber 217 may be rotatable within sheath 219 but constrained from longitudinal translation or entirely constrained from any movement relative to sheath 219. A distal end of imaging probe forms a probe tip 220 that includes a lens assembly 222. Lens assembly 222 is disposed at an end of the optical fiber and includes a micro-lens 223 which is configured to focus light traveling through optical fiber 217 at a distance from lens 223 and to capture a fraction of light that is directed back toward lens assembly 222 for image generation. The particular embodiment depicted is a side-scanning imaging probe such that lens assembly 222 may also include a beam deflector 224, which may be a mirror, offset distally from lens assembly 222 and that may be configured to deflect light 226 passing through lens 223 radially outwardly at a perpendicular angle relative to central axis CA. In other embodiments, lens assembly 222 may be configured to project light 226 radially outwardly at an oblique angle relative to central axis CA or a plurality of angles, such as an oblique angle and the previously mentioned perpendicular angle or a plurality of oblique angles, for example. In a yet further embodiment, probe 212 may be a forward-scanning probe such that deflector 224 is not provided, and light 226 is emitted from probe coincident with the central axis CA to an intended target. Lens assembly 222 may be separately formed and attached to optical fiber 217 or may be molded onto fiber 217.

The light transmitted through optical fiber 217 for generating an OCT image may be in the infrared spectrum. As such, red blood cells and other objects with a red color tend to absorb this light. To help clear red blood cells away from probe tip 220 during image capture and potentially improve image resolution, a saline flush feature 214 may be incorporated into catheter 210. However, flush feature 214 is optional and may be provided in other devices of the system, such as a delivery device 300 described below, for example. The saline flush feature 214 of OCT catheter 210 includes a flush inlet port 215 at a proximal end of catheter 210 for the introduction of saline and one or more flush outlet ports 226 located within probe tip proximate to lens assembly 222 for dispensing saline around probe tip 220. Inlet and outlet ports 215, 226 may be in communication with lumen 218 which facilitates transport of the saline flush from inlet 215 to outlet 226. In another example, saline flush feature 214 may be a liquid contrast feature or used as a liquid contrast feature. In this regard, a liquid contrast agent may be introduced through inlet 215 and emitted through outlet 226 to help enhance fluoroscopic images of fixation device 14 and surrounding tissue in-situ.

In one example, connector 216 forms a proximal interface of OCT catheter 210. Connector 216 is connectable to OCT subsystem 230 which is positioned external to the patient. OCT subsystem 230 may be a controller that controls OCT catheter 210 and/or probe 220. For example, OCT subsystem 230 may be configured to generate and transmit light through OCT catheter 210. In other examples, OCT subsystem 230 may be configured to receive and process light waves returning from probe tip 220. In yet further examples, OCT subsystem may be configured to rotate and/or translate probe tip 220 while in use. Thus, in some examples, OCT subsystem may include one or more drive motors 232. The OCT subsystem 230 may also or alternatively include one or more imaging engines 234. The OCT subsystem 230 may also or alternatively include one or more computing devices 236. The OCT system may also or alternatively include one or more displays 238. These possible components for OCT subsystem 230 are depicted in FIG. 21A. However, it should be understood that OCT subsystem 230 may include any combination of the depicted components and that such components are merely exemplary of the possible components that can be provided in OCT subsystem 230. As such, additional components may be provided in OCT subsystem 230 not mentioned or shown.

Drive motor 232 is connectable to optical fiber 217. Drive motor 232 is configured to rotate and longitudinally translate optical fiber 217 within sheath 219. As such, drive motor 232 may be a single motor with a rotary and linear drive or may be more than one motor such as one rotary drive motor and one linear drive motor, for example. Regardless, drive motor 232 may provide OCT catheter 210 with spin and pull-back functionalities which facilitates the acquisition of 360-degree images along a desired length. In other words, drive motor 232 may be operated so that it is constantly spinning optical fiber 217 within sheath 219 and selectively translates optical fiber 217 proximally-distally (e.g., pullback) within sheath 219 while it is spinning. Since probe 212 directs light radially outwardly, the image that is captured is a 360-degree perspective about central axis CA of probe 212. With the pullback functionality, such 360-degree perspective is extended along a pullback length which can be 10 to 20 mm, for example. Pullback velocity may be up to 40 mm per second in some examples. In other examples, pullback velocity may be about 10 to 20 mm per second. OCT catheter 210 can capture about 180 frames per second for a relatively high resolution as compared to other current technologies, such as ultrasound. Although it is preferable for OCT catheter 210 to have spin and pullback capabilities, OCT catheter 210 may only be provided with a spin functionality such that a 360-degree image generated is at a fixed longitudinal position. In even further embodiments, OCT catheter 210 may have neither spin nor pullback functionality such that the image generated is at a fixed longitudinal location and rotational orientation. In such an embodiment, lens assembly 222 may be configured with a wider-angle view than when spin and pullback functionalities are included.

Imaging engine 234 includes other OCT components commonly utilized to facilitate the operation of OCT catheter 210 and to generate an image signal from light traveling back from probe tip 220. For example, imaging engine 234 may include an interferometer. The interferometer may have several components such as a light source (e.g., laser, laser diode, etc.), a beam splitter, a reflector, and a detector, for example.

Imaging engine 234 may interface with a computing device 236 which may include a processor and a memory. Computing device 236 is connected to display 238 and translates signals received from imaging engine 234, such as from a detector thereof, so that OCT images can be presented to the surgeon in real-time on display 238. Exemplary OCT subsystems that can be alternatively used with OCT catheter 210 are the OPTIS™ Mobile System, OPTIS™ Integrated Next Imaging System, and OPTIS™ Mobile Next Imaging System each with Drive-motor and Optical Controller (DOC), sold by Abbott Vascular, Santa Clara, California, USA. Such systems (e.g., OPTIS Mobile Next Imaging System) may include artificial intelligence that may be trained to identify and highlight (e.g., by applying outlines and/or colors to) structural components of fixation device 14 and leaflet tissue for easier visualization thereof by surgeon. Computing device 236 may also be coupled with one or more other imaging systems, such an angiography system, to further display on display 238 where the OCT images are being captured within the anatomy of the heart.

### C. Delivery Device

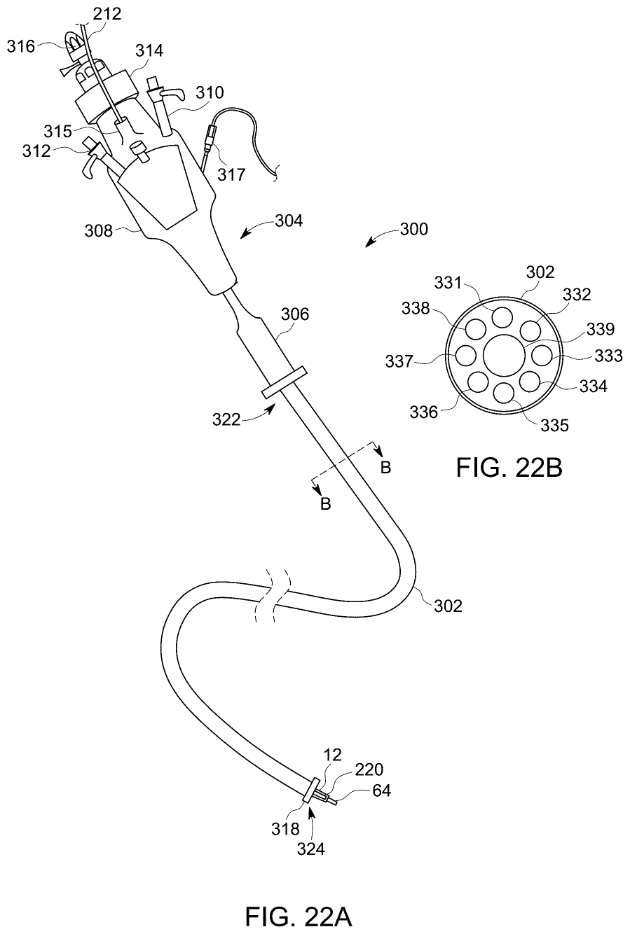

Referring now in addition to FIG. 22A, which provides a perspective view of an embodiment of a delivery device or delivery catheter 300 which may be used to introduce and position a fixation device 14 as described above. Delivery device 300 may also be configured to receive and position OCT probe 212 within fixation device 14, as described in more detail below. In one example, the delivery device 300 includes a shaft 302, having a proximal end 322 and a distal end 324, and a handle 304 attached to the proximal end 322.

Shaft 302 is shown having a nose 318 near its distal end 324. In this embodiment, nose 318 has a flanged shape. Such a flanged shape prevents the nose 318 from being retracted into a guiding catheter or introducer as is described below. However, it should be appreciated that the nose 318 may have any shape including bullet, rounded, blunt or pointed, to name a few. It should also be appreciated that in other embodiments, shaft 302 may not have nose 318 so that shaft 302 can be retracted into and through a guiding catheter. Nose 318 may have openings that correspond to and communicate with a plurality of lumens 331-339 that extend longitudinally through shaft 302. As shown in the cross-sectional view in FIG. 22B, shaft 302 may include up to nine lumens 331-339. Thus, although nine lumens are depicted, shaft 302 may include more or less than nine lumens. In one example, each lumen 331-339 may house a separate control element passing from handle 304 to nose 318 for controlling various mechanisms of fixation device 14.

For example, as previously described in relation to FIGS. 16-19, fixation device 14 may include a locking mechanism 106 which includes a release harness 108. Lock lines 92 connect to release harness 108 to lock and unlock the locking mechanism 106. Lock lines 92 extend from handle 304 through one or more of lumens 331-338 and out through nose 318. Additionally, the raising and lowering of proximal elements 16 is performed through the manipulation of proximal elements lines 90, as previously described. Proximal element lines 90 may also extend from handle 304 through one or more of lumens 331-338 and out through nose 318 where they connect to proximal elements 16. Further, the actuation of distal elements 18 is performed through the manipulation of an actuator rod 64 that is connectable with fixation device 14, such as with stud 74. Actuator rod 64 extends from handle 304 through lumen 339, which is generally centrally positioned within shaft 302, and out from nose 318. A coupling structure, which in this embodiment is shaft 12, extends from distal end 324 of shaft 302 and is configured to couple to coupling member 19 of fixation device 14. Actuator rod 64 extends through shaft 12, as shown in the example of FIG. 22A.

In addition to the aforementioned exemplary control elements 64, 90, 92, shaft 302 of delivery device 300 may also house one or more OCT probes 212 in various examples. For example, an OCT probe 212 may extend from handle 304 through any one of lumens 331-338 such that probe tip 220 extends from nose 318 adjacent to shaft 12 and actuator rod 64, as shown in FIG. 22A. Various arrangements of OCT probe 212 other than that shown in FIG. 22A are described further below. The inclusion of up to nine lumens allows OCT probe 212 to be passed through any one of lumens 331-338 so that probe tip 220 is in a desired position relative to fixation device 14 while also allowing the control elements to be positioned as needed. Also, as previously mentioned, it may be beneficial to provide a saline flush proximate to lens assembly 222 to aid in image capture. As shown in FIG. 22A, a flush inlet port 317 may be provided in handle 304. Such inlet port 317 may be in communication with one or more lumens 331-338 so that saline flush can be projected out of nose 318 toward a fixation device 14 connected to shaft 302.

Handle 304 is attached to the proximal end 322 of the shaft 302. Handle 3010 is used to manipulate the coupled fixation device 14 and to optionally decouple the fixation device 14 for permanent implantation. In this regard, one example of handle 304 generally includes an actuator rod control 314, an actuator rod handle 316, a lock line handle 310, and a proximal element line handle 312. As described, the fixation device 14 is primarily manipulated by the actuator rod 64, proximal element lines 90, and lock lines 92. In one example, the actuator rod 64 manipulates the distal elements 18, the proximal element lines 90 manipulate the proximal elements 16, and the lock lines 92 manipulate the locking mechanism 106. The actuator rod 64 may be translated (extended or retracted) to manipulate the distal elements 18 from handle 304 according to one example. This is achieved with the use of the actuator rod control 314, for example. The actuator rod 64 may also be rotated to threadedly engage or disengage the threaded stud 74 of fixation device 14. This is achieved with the use of the actuator rod handle 316. Further, the proximal element lines 90 may be extended, retracted, loaded with various amounts of tension or removed with the use of the proximal element line handle 312. The lock lines 92 may be extended, retracted, loaded with various amounts of tension or removed with the use of the lock line handle 310. The actuator rod handle 316, actuator rod control 314, proximal element line handle 312 and lock line handle 310 are all joined with a main body 308 within which the actuator rod 64, proximal element lines 90 and lock lines 92 are guided into the shaft 302 according to one example. The example handle 304 further includes a support base 306 connected with the main body 308. In one example, the main body 308 is slideable along the support base 306 to provide translation of the shaft 302. Further, in one example, the main body 308 is rotatable around the support base 306 to rotate shaft 302. Although only one lock line handle 310 and proximal element handle 312 are shown, it should be appreciated that more than one of each handle 310, 312 may be provided.

Handle 304 may also include an OCT probe interface 315 that provides an opening that allows for the insertion of OCT probe 212 through handle 304 and into a corresponding lumen 331-338 of shaft 302 according to one example. As mentioned, more than one OCT probe 212 may be utilized with delivery device 300. As such, more than one OCT probe interface 315 may be provided. Although not shown, a hemostatic valve may be provided at the OCT probe interface 315 to prevent back bleeding and reduce the possibility of air introduction when inserting OCT probe 315 into handle 304.

### D. Guide Catheter Assembly

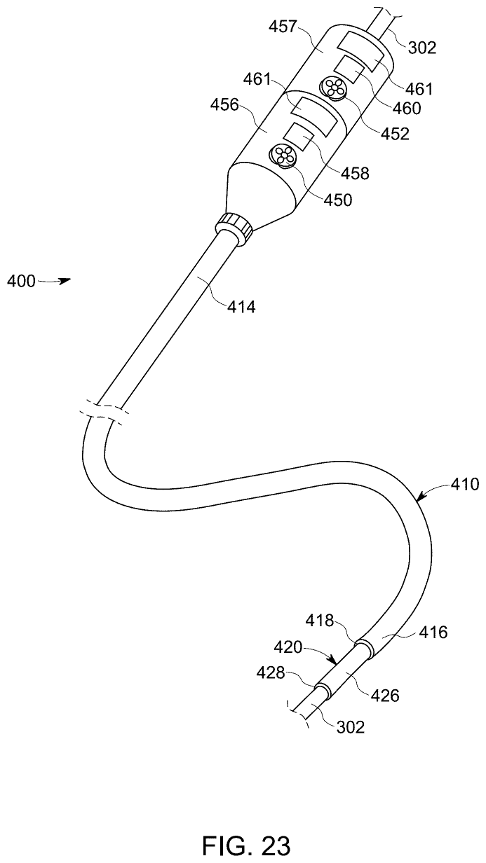

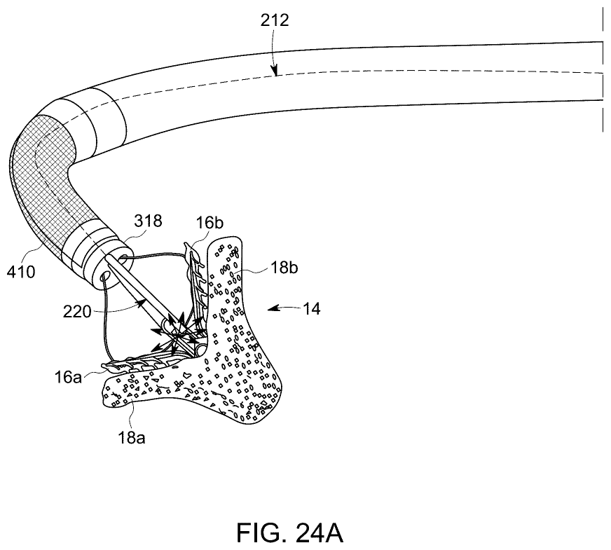

Referring now in addition to FIG. 23, which depicts an embodiment of a multi-catheter guiding system or guide catheter assembly 400 of the present disclosure. System 400 comprises an outer guide catheter 410, having a proximal end 414, a distal end 416, and a central lumen 418 therethrough. System 400 also comprises an inner guide catheter 420, having a proximal end 424, distal end 426 and central lumen 428 therethrough. The inner guide catheter 420 is positioned coaxially within the central lumen 418 of the outer guide catheter 410, as shown. The distal ends 416, 426 of catheters 410, 420, respectively, are sized to be passable to a body cavity, typically through a body lumen such as a vascular lumen. Thus, in various examples, the distal end 416 preferably has an outer diameter in the range of approximately 0.040 in. to 0.500 in. (1.02 mm to 12.7 mm), more preferably in the range of 0.130 in. to 0.320 in. (3.30 mm to 8.13 mm). In various examples, central lumen 418 is sized for the passage of the inner guide catheter 420; the distal end 426 preferably has an outer diameter in the range of approximately 0.035 in. to 0.280 in. (0.89 mm to 7.11 mm), more preferably 0.120 in to 0.200 in. (3.05 to 5.08 mm). The central lumen 428 is sized for the passage of a variety of devices therethrough, such as shaft 302 of delivery device 300 in one example. Therefore, the central lumen 428 preferably has an inner diameter in the range of approximately 0.026 in. to 0.450 in. (0.66 mm to 11.43 mm), more preferably in the range of 0.100 in. to 0.180 in. (2.54 mm to 4.57 mm).

The outer guide catheter 410 and/or the inner guide catheter 420 are precurved and/or have steering mechanisms to position the distal ends 416, 426 in desired directions according to various examples. Precurvature or steering of the outer guide catheter 410 directs the distal end 416 in a first direction to create a primary curve while precurvature and/or steering of the inner guide catheter 420 directs distal end 426 in a second direction, differing from the first, to create a secondary curve. Together, the primary and secondary curves form a compound curve in one example. As shown, shaft 302 of delivery device 300 may be advanced through and guided by guide catheters 410, 420. Advancement of delivery device shaft 302 through the coaxial guide catheters 410, 420 guides shaft 302 through the compound curve toward a desired direction, usually in a direction which will allow distal end 324 of shaft 302 and a fixation device 14 connected thereto to reach its target, such as a mitral or tricuspid valve.

Steering of the outer guide catheter 410 and inner guide catheter 420 may be achieved by actuation of one or more steering mechanisms. Actuation of the steering mechanisms is achieved with the use of actuators which are typically located on handles connected with each of the catheters 410, 420 in some examples. As illustrated in FIG. 23, handle 456 is connected to the proximal end 414 of the outer guide catheter 410 and remains outside of the patient's body during use. Handle 456 includes steering actuator 450 which may be used to bend, arc or reshape the outer guide catheter 410, such as to form a primary curve. Handle 457 is connected to the proximal end (not shown) of the inner guide catheter 420 and may optionally join with handle 456 to form one larger handle, as shown. Handle 457 includes steering actuator 452 which may be used to bend, arc, or reshape the inner guide catheter 420, such as to form a secondary curve and move the distal end 426 of the inner guide catheter 420 through desired angle.

In addition, in some examples, locking actuators 458, 460 may be used to actuate locking mechanisms to lock the catheters 410, 420 in a particular position. Actuators 450, 452, 458, 460 are illustrated as buttons, however it may be appreciated that these and any additional actuators located on the handles 456, 457 may have any suitable form including knobs, thumbwheels, levers, switches, toggles, sensors, or other devices.

In addition, in some examples, handle 456 may include a numerical or graphical display 461 of information such as data indicating the position the catheters 410, 420, or force on actuators. It may also be appreciated that actuators 450, 452, 458, 460 and any other buttons or screens may be disposed on a single handle which connects with both the catheters 410, 420.

FIG. 23 also illustrates that shaft 302 of delivery device 300 may extend through handles 456 and 457 and within inner guide catheter 420 in some examples. Shaft 302 may extend distally from distal ends 426, 428 of inner and outer guide catheters 416, 426 which allows a fixation device 14 connected to distal end 324 of shaft 302 and an OCT probe 220 extending from shaft 302 to be positioned within a target valve according to examples of the disclosure. As mentioned above, shaft 302 may include a nose 318 in the form of a flange that forms a stop in one example. Such stop prevents distal end 324 of shaft 302 from entering the central lumen 428 of the inner guide catheter 420. Thus, shaft 302 may be advanced and retracted until nose 318 contacts the distal end 426 of the inner guiding catheter 420 preventing further retraction. This may provide certain advantages during some procedures. It may be appreciated that in embodiments which include such a stop 318, shaft 302 would be pre-loaded within the inner guide catheter 420 for advancement through the outer guiding catheter 410 or both the shaft 302 and the inner guiding catheter 420 would be pre-loaded into the outer guiding catheter 410 for advancement to the target valve. This is because nose 318 prevents advancement of shaft 318 through the inner guiding catheter 420. However, in other embodiments of shaft 302 in which nose 318 is not included, distal end 324 may be retracted into inner guiding catheter 420. This may allow shaft 302 to be withdrawn from guide catheter assembly 400 while assembly 400 remains in place within the vasculature of the patient. As such, another delivery device 300 or the same delivery device 300 with another fixation device 14 connected thereto can be guided to the same target valve for the deployment of the additional fixation device 14.

### F. OCT Probe Arrangements

When in use in conjunction with fixation device 14, OCT probe 212 can take on any one of a variety of different arrangements relative to fixation device 14. Such arrangements allow a surgeon to use OCT catheter system 200 to observe and/or peer into fixation device 14 during a procedure to assess leaflet capture and/or security.

i. Central Placement

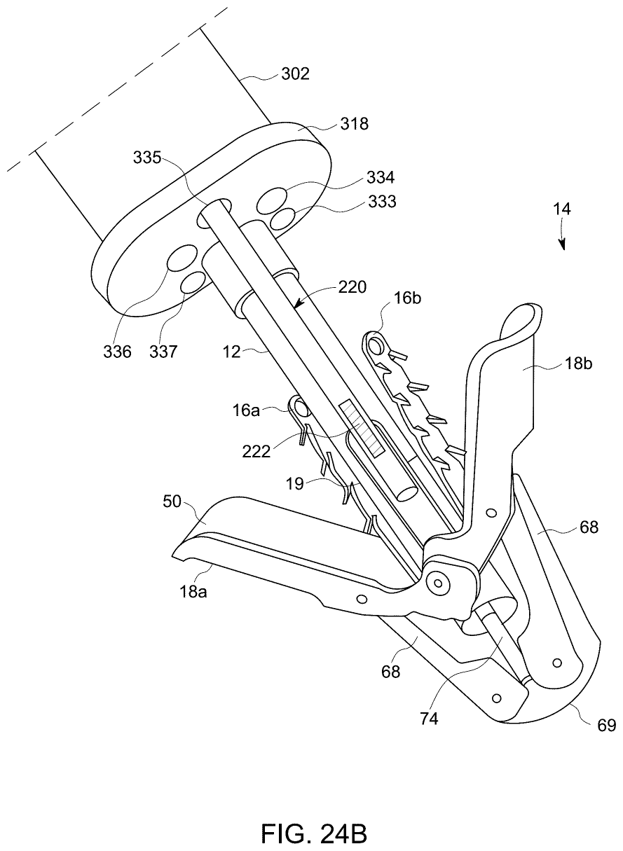

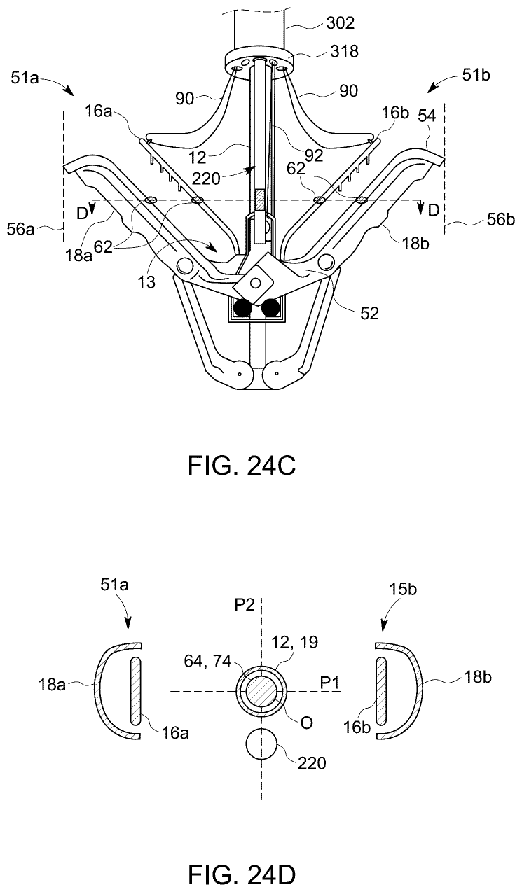

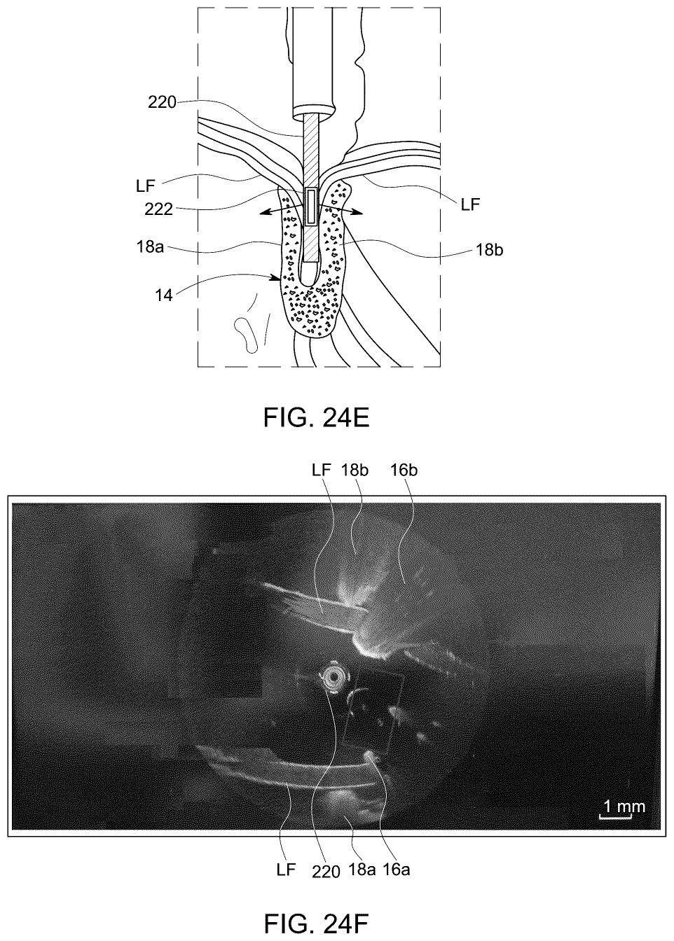

Referring now in addition to FIGS. 24A-24F, which depict an example of a first central arrangement of OCT imaging probe 212 relative to fixation device 14. In one example, fixation device 14 includes a first clamp 51a (or first clip 51a) comprised of first proximal and distal elements 16a, 18a and a second clamp 51b (or second clip 51b) comprised of second proximal and distal elements 16a, 18b. In one example, first distal element 18a defines a first lateral extent of fixation device. Second distal element 18b defines a second lateral extent of fixation device 14. This is shown in FIG. 24C. In some examples, imaging probe 212 extends through delivery device 300 and distally from shaft 302, such as from lumen 335, so that probe tip 220 is positioned between the lateral extents of fixation device 14. More specifically, in some examples, probe tip 220 is positioned centrally between clamps 51a-51b adjacent and substantially parallel to a center body (or center portion) of fixation device 14. In the embodiment depicted, fixation device 14 may include coupling member 19 and stud 74, as best shown in FIG. 24B.

FIG. 24D is a cross-sectional schematic taken at the 50% leaflet insertion depth and further illustrates this relationship within a Cartesian coordinate system established by a first plane P1 and a second plane P2. First plane P1 is orthogonal to second plane P2, and each plane P1, P2 bisects shaft 12, actuator rod 64, and coupling member 19. This intersection defines the origin O. The origin O is also generally the center of fixation device 14 and coincides with a central axis of shaft 12, actuator rod 64, and coupling member 19. First plane P1 also intersects first and second clamps 51a-b. Second plane P2, on the other hand, lies substantially equidistant from each clamp 51a-b.

In this example first central arrangement, probe tip 220 is intersected by second plane P2 such that probe tip 220 is positioned equidistant from clamps 51a-51b. However, since shaft 12 and coupling member 19 occupy the origin O, probe tip 220 is positioned offset from first plane P1. In other words, probe tip 220 is oriented 90 degrees in a clockwise direction, about the origin O, from first plane P1. In this example, lens assembly 222 may be positioned at a height or location that corresponds to the 50% insertion depth of clamps 51a-51b. In this regard, an OCT cross-sectional image can be generated by OCT catheter system 200 at this height which would allow a surgeon to assess whether leaflet tissue in any one of clamps 51a-51b has reached beyond this critical depth. FIG. 24F depicts such a cross-sectional image at the 50% leaflet insertion depth. As illustrated, proximal and distal elements 16a-16b, 18a-18b of first and second clamps 51a-51b are visible within the OCT image. Even further, leaflet tissue is visible between first and second clamps 51a-51b thereby indicating that a depth beyond the 50% leaflet insertion depth threshold has been achieved. The light used by imaging probe 212 to generate the OCT image penetrates leaflet tissue with sufficient depth that leaflet thickness at this insertion depth can be measured. Thus, a surgeon can assess the quality of the tissue being grasped and the depth of leaflet insertion to ensure that the leaflets have been sufficiently secured by clamps 51a-51b.

It should be appreciated that clamps 51a-51b can rotate to various positions with respect to the central axis of fixation device which may affect the height at which lens assembly 222 should be located to be at the 50% leaflet insertion depth. However, leaflet capture typically occurs at a 60-degree orientation and therefore the lens assembly position can be determined based on this 60-degree orientation. Nonetheless, lens assembly 222 need not be fixed at a particular height. As previously described, imaging probe 212 may have a pullback functionality such that lens assembly 222 can be shuttled longitudinally along the length of shaft 12 and coupling member 19. In this regard, a three-dimensional, 360-degree image from within fixation device 14 can be generated by OCT catheter system 200 allowing the surgeon to visualize the entire depth of leaflet insertion and the structure of the tissue being grasped. Additionally, each proximal element 16a-16b and/or distal element 18a-18b may include a reflector 62 disposed at the 50% leaflet insertion depth, as best shown in FIG. 24C. Such reflectors 62 may provide a visual reference in the OCT image for comparison to the depth of the leaflets in some examples. As such, if the leaflet depth does not surpass the 50% leaflet insertion depth indicated by reflectors 62, then the surgeon may determine to attempt a regrasp of the leaflet according to an example of the disclosure. Thus, at least one advantage of the use of OCT system 200 with fixation device 14 is the ability of a surgeon to create a direct prompt for when a leaflet is to be regrasped based on the OCT image generated by system 200.

In operation, OCT probe catheter 210 may be pre-coupled to delivery device 300 in relation to fixation device 14 or may be assembled with delivery device in the operating theater during the procedure either before or after introduction of fixation device 14 into a patient's heart. Once imaging probe tip 212 is in the desired arrangement, optical fiber 217 is rotated and/or translated about the central axis CA of imaging probe 212. Light 226 is emitted radially outwardly toward first and second clamps 51a-51b and valve leaflets. Reflected light is captured by probe tip 220 and transmitted proximally through optical fiber 217 to OCT subsystems 230 where such light is used to generate a signal which is converted to a real-time image displayed on display 238. If it is determined that a valve leaflet is not within one of the clamps 51a-51b, then the surgeon may, in one example, attempt to regrasp the leaflet until a depth beyond the 50% leaflet depth is achieved.

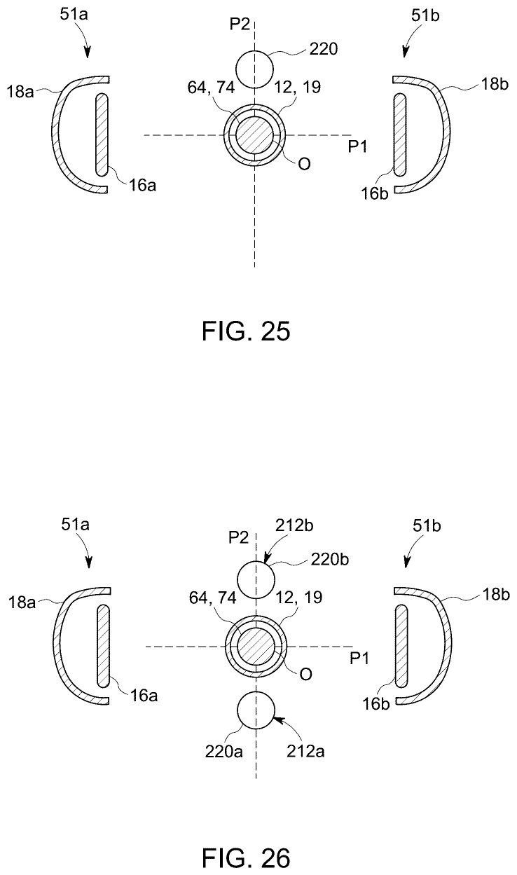

Referring now in addition to FIG. 25, which depicts an example of a second imaging probe central arrangement between the lateral extents of fixation device 14. This arrangement is generally the same as the first arrangement described above with the difference being that probe tip 220 is disposed at the opposite side of first plane PI and of shaft 12/coupling member 19. In other words, in one example, probe tip 220 is oriented 90 degrees counterclockwise (i.e., 270 degrees clockwise), about the origin O, relative to first plane P1. As such, probe tip 220 is intersected by second plane P2 and is offset from first plane P1 along second plane P2.

Referring now in addition to FIG. 26, which depicts an example of a dual imaging probe central arrangement. In this example arrangement, first and second probe tips 220a-220b are each intersected by second plane P2 and are positioned at opposite sides of shaft 12 offset from first plane P1. Thus, first probe 220a is located in the same position as the first central arrangement described above, and second probe 220b is located at the same position as the second central arrangement described above. As such, in this example, the first probe tip 220a is oriented 180 degrees with respect to second probe tip 220b. While imaging probes 212a-212b may each be used to generate their own image from their relative perspectives within fixation device 14, such images can be integrated together by computing device 236 and displayed as a single image on display 238. Also, although a single imaging probe 212 may be sufficient to visualize both leaflets, because shaft 12 is positioned at the origin O of first and second planes P1, P2, shaft 12 may obstruct a segment of any one probe's field of view. The use of probes 212a-212b can account for this obstruction so that the full 360 degree perspective can be obtained.

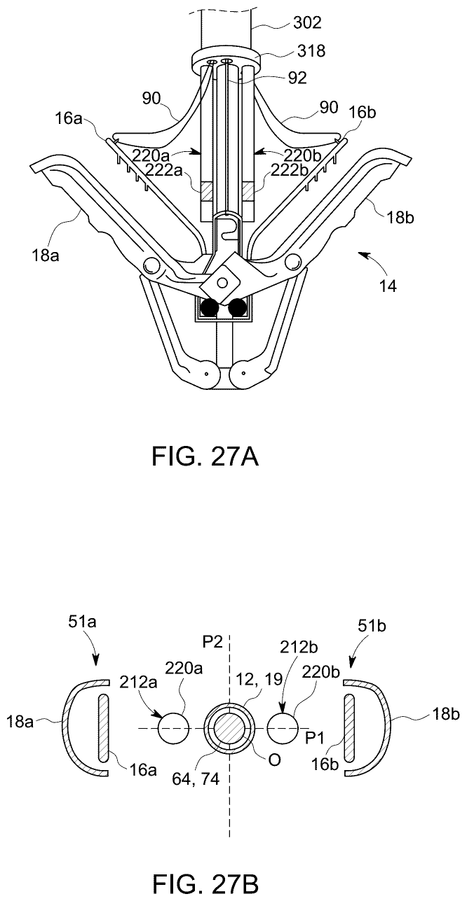

Referring now in addition to FIGS. 27A and 27B, which depict another dual probe central arrangement embodiment. In this example arrangement, first and second imaging probes 220a-220b are each intersected by first plane P1 and are positioned offset from second plane P2. In other words, first probe tip 220a is oriented 90 degrees clockwise, about the origin O, relative to P2, second imaging probe 220b is oriented 90 degrees counterclockwise, about the origin O, relative to P2, and probes 220a-b are oriented 180 degrees relative to each other. Thus, first probe tip 220a is positioned closer to first clamp 51a than is the second probe tip 220b, and second probe tip 220b is positioned closer to second clamp 51b than is the first probe tip 220a. Such an example arrangement may facilitate a higher resolution as the respective lens assemblies 222 of first and second probes 212a-212b are generally closer to their respective clamps 51a-51b than in the other arrangements previously described. However, since such probe tips 220a-220b are positioned directly between proximal elements 16a-16b and shaft 12, fixation device 14 may not be able to be completely closed as it is advanced through guide catheter assembly 400. Alternatively, in some examples, probes 212a-212b may be introduced through their respective lumens, such as lumens 337 and 333, after fixation device 14 is positioned at the target valve and proximal elements 16a-16b are moved to a position to provide clearance for probe tips 220a-220b.

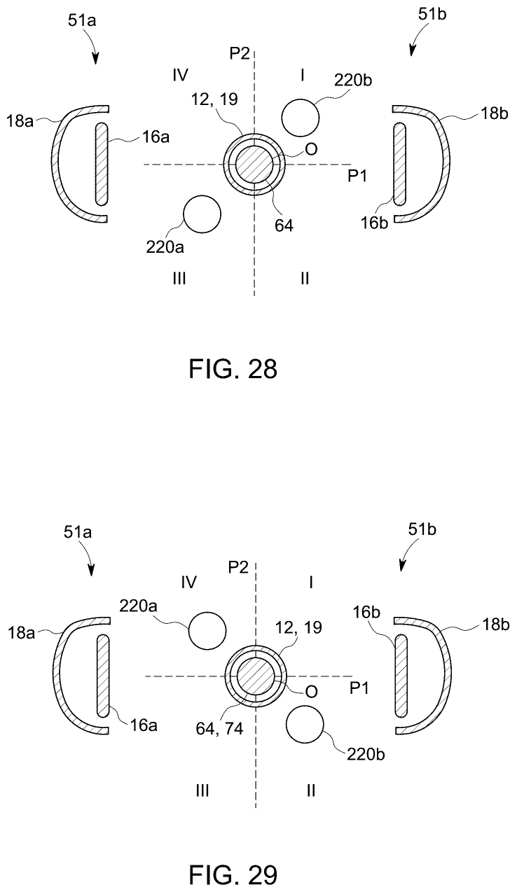

Referring now in addition to FIGS. 28 and 29, which depict even further dual probe central arrangement embodiment examples. First and second planes P1, P2 define four quadrants I-IV about shaft 12 such that the first and second quadrants I, II are closest to second clamp 51b, and the third and fourth quadrants are closest to first clamp 51a. In these arrangements, first and second probe tips 220a-220b may extend from respective lumens of shaft 302 so that they are positioned within respective quadrants.

For example, as shown in FIG. 28, first probe tip 220a is located in the third quadrant III, while second imaging probe 220b is located in the first quadrant I. More specifically, first probe tip 220a is oriented about 45 degrees clockwise, about the origin O, with respect to P2, while second probe tip 220b is oriented at about 45 degrees counterclockwise, about the origin O, from P1. Thus, first probe tip 220a is positioned closer to first clamp 51a, while second imaging probe 220b is positioned closer to second clamp 51b. It should be appreciated though that imaging probes 220a-220b can occupy any angle between Pl and P2 within their respective quadrants. However, it is preferable that probe tips 220a-220b are oriented 180 degrees with respect to each other. The other example shown in FIG. 29 is essentially the same as the example just described except that first probe tip 220a is positioned within the fourth quadrant IV, while second probe tip 220b is positioned in the second quadrant II. Either of these arrangements may be selected to position probe tips 220a-220b closer to respective clamps 51a-51b, while being positioned to avoid interfering with proximal elements 16a-16b. It is also contemplated that a single probe 212 may be positioned in any one of the quadrants I-IV rather than utilizing a dual probe configuration provided that shaft 12 does not interfere with the probe's ability to show the captured status of tissue at the far side of probe 212.

While in each of the aforementioned dual probe arrangements, first and second probe tips 220a-220b are depicted at the same height relative to shaft 12/coupling member 19, it should be appreciated that probe tips 220a-220b may be positioned at different heights so that their respective lens assemblies 222 can separately image clamps 51a-51b and valve leaflets at such heights. For example, first probe 212a may be positioned so that a lens assembly 222 thereof is positioned equal to or greater than 50% leaflet insertion depth (e.g., 50% to 75%), while second probe tip 220b may be positioned so that a lens assembly 222 thereof is positioned at less than 50% leaflet insertion depth (e.g., 25% to 45%). This may allow a surgeon to observe a plurality of leaflet depths without the use of a pullback feature. This may be useful in circumstances where tortuous pathways throughout the vasculature restrict translational movement of an optical fiber 217.

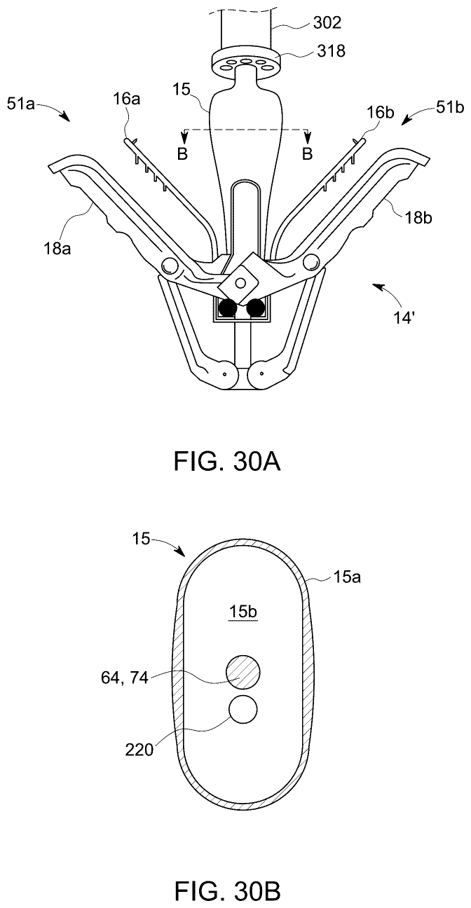

Referring now in addition to FIGS. 30A and 30B, which depict an example fixation device 14′ that includes a center body that is a central spacer 15. In one example, central spacer 15 is positioned between first and second clamps 51a-51b. Central spacer 15 may have an ovular cross-sectional shape, as shown in FIG. 30B. Central spacer may taper inwardly along a longitudinal direction, as shown in FIG. 30A. However, central spacer 15 can have other shapes, such as a teardrop shape, for example. Spacer 15 has a shell or sidewall 15a that defines an interior space 15b. Interior space 15b includes actuator rod 64 for actuating distal elements 18a-18b. One or more probe tips 220 may also be disposed within this interior space 15b. For example, as shown in FIG. 30B, a single probe tip 220 is positioned adjacent to and generally parallel to actuator rod 64. While shown in the same position relative to actuator rod 64 as the first central arrangement described above, it should be understood that probe tip 220 may be located in any one of the other positions also described above, although within spacer shell 15a. It should also be appreciated that a second probe tip may be positioned within spacer 15 in any one of the positions described above with respect to the double probe arrangements. Central spacer 15 may be made from a biocompatible polymer material that is translucent to the wavelength of light emitted from probe tip 220 such that it would not interfere with the transmission of such light. After fixation device 14′ is affixed to valve tissue, actuator rod 64 and/or imaging probe 212 may be removed from interior space 15b.

ii. Distal Element Placement Examples

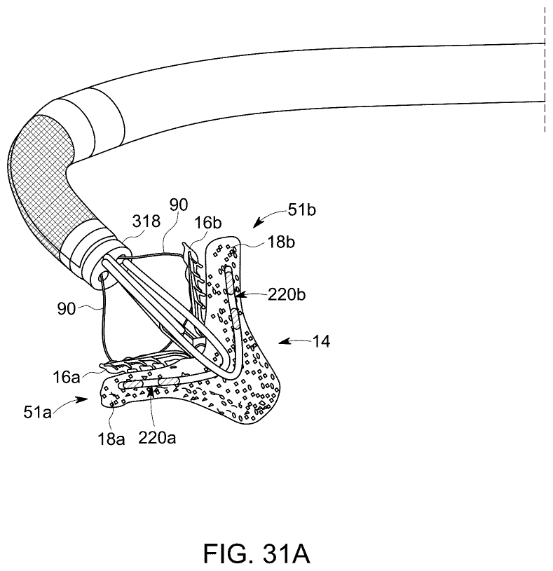

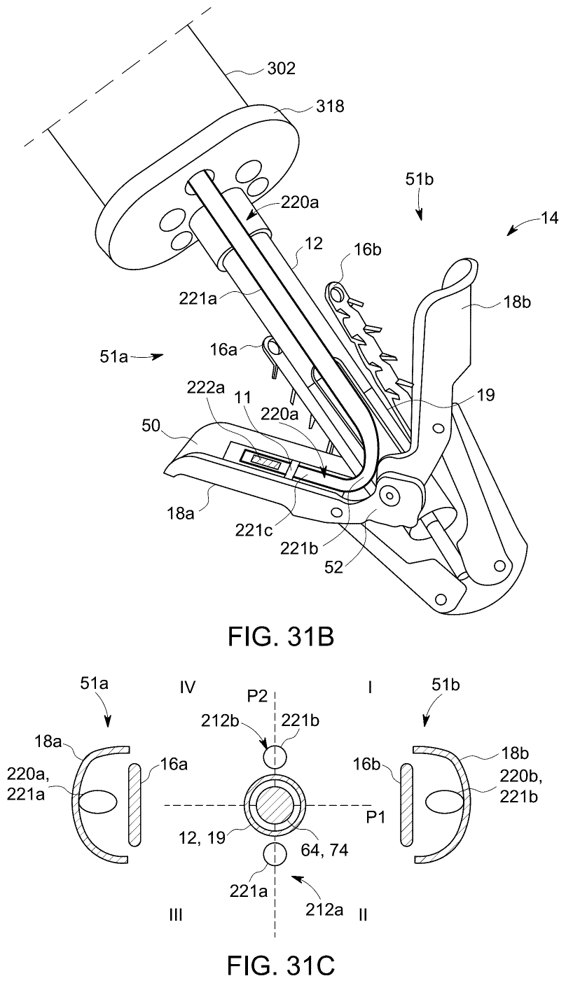

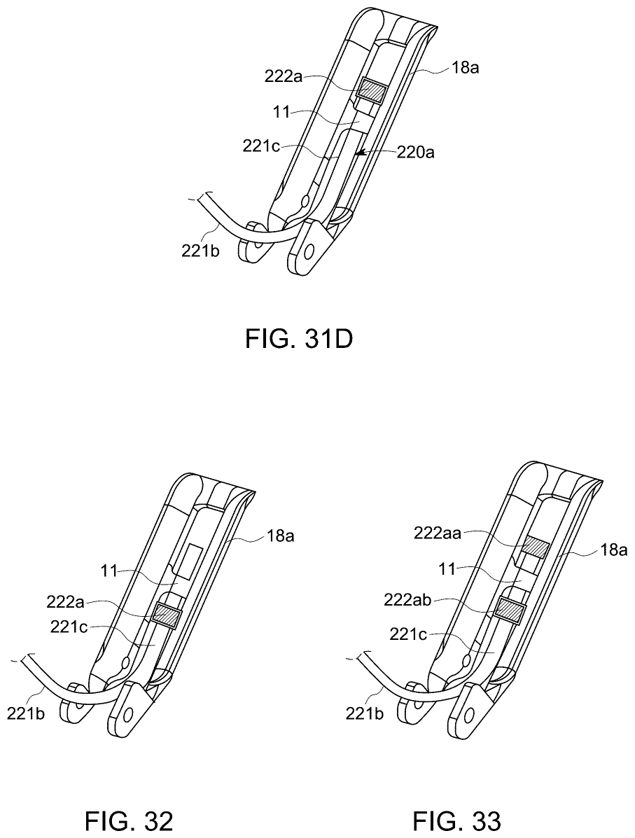

Referring now in addition to FIGS. 31A-31D that depict a dual imaging probe clamp arrangement with respect to fixation device 14. In this arrangement, first and second imaging probe tips 220a-220b are positioned between lateral extents of fixation device 14. However, instead of being centrally arranged, probe tips 220a-220b are positioned within first and second clamps 51a-51b, respectively. More specifically, first imaging probe 212a is routed such that first probe tip 220a is positioned within first distal element 18a between first proximal and distal elements 16a, 18a, and second imaging probe 220b is routed such that second probe tip 212b is positioned within second distal element 18b between second proximal and distal elements 16b, 18b.

As best shown in FIGS. 31A and 31D, first imaging probe 221a has a first straight segment 221a, a second straight segment 221c, and a curved segment 221b between the first and second straight segments 221a, 221c. First straight segment 221a extends from a lumen of shaft 302. Such lumen may be lumen 334 or 335, for example. First straight segment 221a extends adjacent to and along a length of shaft 12/coupling member 19 toward a distal end 52 of first distal element 18a. Curved segment 221b extends from a distal end of first straight segment 221a and curves toward engagement surface 50 of distal element 18a. Second straight segment 221c, which includes probe tip 220a, extends from curved segment 221b and along engagement surface 50 in a proximal direction. In this regard, probe tip 220a lies along a majority of the length of distal element 18a and parallel to engagement surface 50. Second straight segment 221c is routed between engagement surface 50 and a crossbar 11 extending across engagement surface 50 which helps retain straight segment 221c within distal element 18a. In other embodiments, second straight segment 221c may instead be embedded within covering 100 (see FIG. 15A) encompassing the entire length of distal element 18a.

Crossbar 11 may be located at the 50% leaflet insertion depth for first clamp 51a. As shown in FIGS. 31A and 31D, lens assembly 222a may be positioned and fixed proximal to crossbar 11. The positioning of lens assembly 222a at this position relative to the crossbar 11 allows a surgeon to determine whether a depth beyond the 50% leaflet insertion depth has been achieved. In another embodiment shown in FIG. 32, lens assembly 222a may be positioned and fixed distal to crossbar 11. In a further embodiment shown in FIG. 33, a first lens assembly 222aa positioned proximal to crossbar 11, and a second lens assembly 222ab positioned distal of crossbar 11. Alternatively, first and second lens assemblies 222aa-222ab may be provided on two separate imaging probes routed into first distal element 18a.

Although in some embodiments lens assembly 222a may be in a fixed position within first distal element 18a, other embodiments of first imaging probe 212a may have a pullback functionality. In such embodiments, optical fiber 217 of first imaging probe 212a may have a minimal diameter and/or made with a flexible material to reduce bending stresses and to facilitate rotational and translational movement of optical fiber 217 within sheath 219 to help overcome the tight curvature of curved segment 221b. Additionally, the lumen of shaft 302 through which first straight segment 221a extends may be selected to provide the largest radius of curvature possible to first curved segment 221b which may further facilitate pullback. For first imaging probe 212a, this may be lumen 335 which lies along second plane P2, as shown in FIG. 31C, or lumen 334 which is located in second quadrant II offset from P1 and P2. When pullback is provided, lens assembly 222a may translate along a majority of the length of first distal element 18a. This may allow a surgeon to view the precise depth at which a leaflet is captured. The crossbar 11 may appear in the OCT image as a metallic artifact indicating to the surgeon the 50% leaflet insert depth threshold and allowing the surgeon to determine that the leaflet has been inserted beyond this threshold depth. Alternatively, a reflector, such as reflector 62, may be provided to indicate the critical depth.

It should be understood that second imaging probe 212b may be configured the same as first imaging probe 212a and extends into second distal element 18b in the same manner just described with relation to first imaging probe 212a. Alternatively, the second imaging probe may be configured as in one of the central placement arrangements, to provide an alternative visualization perspective.

iii. Proximal Element Placement

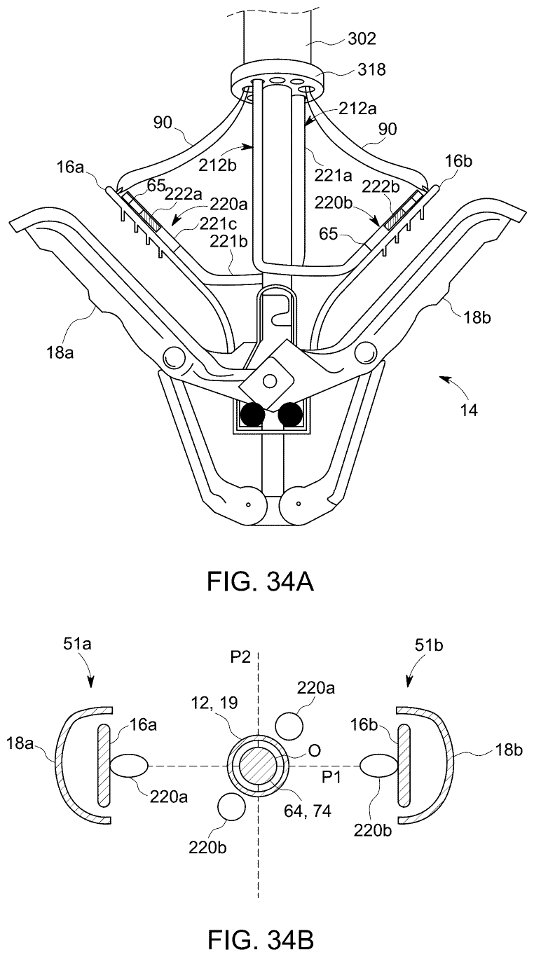

Referring in addition to FIGS. 34A and 34B, which depict another example of dual probe clamp arrangement. In this arrangement, first and second imaging probe tips 220a-220b are positioned between lateral extents of fixation device 14. However, instead of probe tips 220a-220b being disposed within clamps 51a-51b, they are disposed external to clamps 51a-51b and on proximal elements 16a-16b, respectively. More specifically, first imaging probe 212a is routed such that first probe tip 220a is positioned along a proximal side of first proximal element 16a, and second imaging probe 220b is routed such that second probe tip 212b is positioned along second proximal element 16b. Similar to the embodiment described above with respect to FIGS. 31A-31D, first imaging probe 212a may have a first straight segment 221a, a second straight segment 221c, and a curved segment 221b extending therebetween. First straight segment 221a extends from delivery device shaft 302 toward a distal end of fixation device 14. Curved segment 221b extends from a distal end of first straight segment 221a toward first proximal element 16a. Second straight segment 221c extends from curved segment 221b and along a proximal side of first proximal element 16a and may secured thereto via eyelets 65 or a covering that covers element 16a, for example. A first lens assembly 222a may be fixed at a 50% leaflet insertion depth. However, in other embodiments, pullback may be provided so that lens assembly 222a traverses the majority of the length of first proximal element 16a. Second imaging probe 212b is similarly configured and extends along the proximal side of second proximal element 16b in the same manner just described with relation to first imaging probe 212a. Alternatively, the second imaging probe may be configured as in one of the central placement arrangements or as in the distal element placement, to provide an alternative visualization perspective.

iv. Continuous Monitoring and Advance Scouting

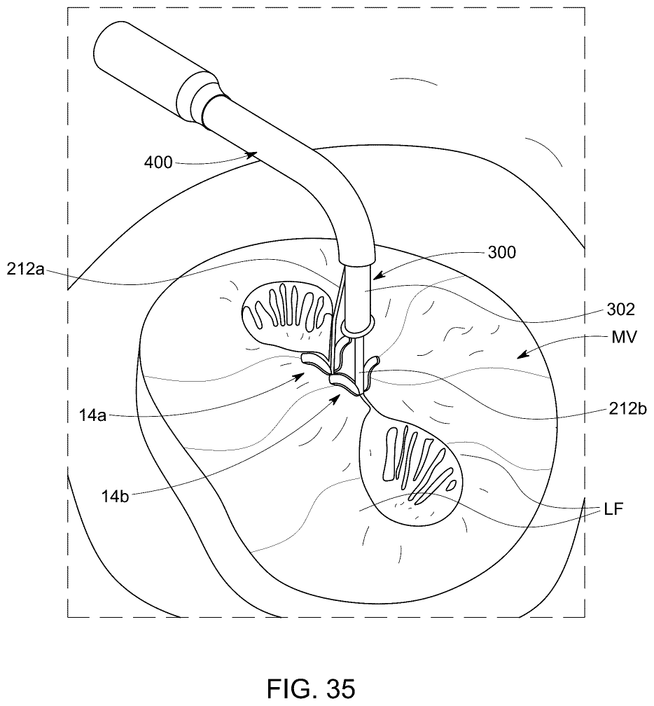

As illustrated in FIG. 35, which is referenced in addition to the aforementioned figures, some edge-to-edge repair procedures may involve the implantation of more than one fixation device 14. After the implantation of a first fixation device 14a using OCT imaging, it may be desirable to continue to monitor first fixation device 14a with a first imaging probe 212a while a second fixation device 14b is implanted. FIG. 35 depicts an embodiment of the system that facilitates such continued monitoring. In this embodiment, a first imaging probe 212a may extend through a lumen in guide catheter assembly 400 rather than through shaft 302 of delivery device 300. Although first imaging probe 212a may extend through a lumen in guide catheter assembly 400, first imaging probe 212a may still be positioned in any one of the aforementioned relationships with respect to first fixation device 14a. Once first fixation device 14a is implanted, delivery device 300 may be withdrawn from guide catheter assembly 400 while first imaging probe 212a remains in place within fixation device 14a. Another delivery device 300 or the same delivery device 300 with a second fixation device 14b connected thereto may be reintroduced to guide catheter assembly 400 for subsequent implantation. To the extent guide catheter assembly 400 may need to be repositioned for placement of second fixation device 14b, first imaging probe 212a may flex as assembly 400 is moved to prevent probe 212a from being inadvertently removed from first fixation device 14a. A second imaging probe 212b may then be used to assist the implantation of second fixation device 14b and, in such circumstance, may extend through delivery device 300 as in any of the embodiments previously described.

It may also be appreciated that first imaging probe 212a can provide advance scouting and diagnostics ahead of implantation of first fixation device 14a. For example, first imaging probe 212a or a separate imaging probe may be advanced from guide catheter assembly 400 to the target valve prior to advancing first fixation device 14a from guide catheter assembly 400. In this regard, first imaging probe 212a may be advanced to a position between leaflets LF where an OCT scan may be performed. This initial scan may help a surgeon identify an ideal location for implantation and assess leaflet quality before proceeding with the procedure. A forward-scanning or side-scanning imaging probe may be utilized for this purpose.

### G. System Advantages

The fixation systems with leaflet capture assessment described herein provide numerous benefits over current known techniques and systems. In this regard, the use of imaging probes with a fixation device, as described, allows a surgeon to obtain high resolution two-dimensional and three-dimensional images from within the fixation device and to observe the valve leaflets as they are grasped. Leaflet thickness can also be measured. The quality of the tissue being grasped can also be assessed. Thus, surgeons can develop direct diagnostic prompts around this technology that simplifies decision making during the surgical procedure.

Moreover, the OCT approaches detailed herein are much more accurate than other modalities, such as ultrasound. For example, current OCT technology is capable of resolving features/depths at a resolution of 20 μm, which is far better at resolving small features than traditional intravascular ultrasound (70 to 200 μm). In addition, OCT device construction is much simpler than intravascular ultrasound and can therefore accommodate a much smaller probe profile (e.g., 3 French or less). Furthermore, OCT imaging technology is non-electrical within the patient as light is passively transmitted through an optical fiber thereby negating any need for electrical isolation and any concerns of introducing an electrically active component into an electrically active heart. The OCT technology contemplated herein can also enhance the performance of tricuspid valve repair and overcome the additional limitations of TEE for such surgical intervention.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A fixation system for engaging tissue of a patient, comprising:
a delivery device having a shaft defining a lumen extending from a first end to a second end of the shaft;
an implantable fixation device having a first clamp, a second clamp, and a center portion connected to and extending between the first and second clamps, the center portion being releasably connected to a distal end of the shaft of the delivery device, the first clamp defining a first lateral extent of the fixation device, and the second clamp defining a second lateral extent of the fixation device; and
a first optical coherence tomography (“OCT”) catheter configured for cardiovascular imaging having a first imaging probe comprising a first end, a second end, and a first lens assembly disposed at the second end of the first imaging probe, the first imaging probe being configured to transmit light between the first and second ends thereof,
wherein, in an assembled condition of the fixation system, the first imaging probe extends through the lumen and out from the second end of the shaft such that the first lens assembly is positioned between the first and second lateral extents of the fixation device.

2. The system as in claim 1, wherein the first imaging probe includes a sheath and an optical fiber disposed within the sheath, the first lens assembly being disposed at an end of the optical fiber.

3. The system as in claim 2, wherein the optical fiber is rotatable and translatable within the sheath.

4. The system as in claim 1, wherein the OCT catheter includes a flush feature having an inlet port at the first end of the first imaging probe and an outlet port at the second end of the first imaging probe.

5. The system as in claim 1, wherein the first lens assembly includes a lens and a beam deflector, the beam deflector being configured to deflect the light transmitted through the first imaging probe in a direction radially outwardly from a central axis of the imaging probe.

6. The system as in claim 5, wherein the beam deflector is configured to deflect the light at a perpendicular angle relative to the central axis.

7. The system as in claim 1, wherein the delivery device includes a handle connected to the first end of the shaft, the handle having an OCT probe interface configured to receive the first imaging probe and direct it into the lumen of the shaft.

8. The system as in claim 7, wherein the lumen is a first lumen of a plurality of lumens, and the delivery device further includes an actuator rod extending through a second lumen of the plurality of lumens and out from the second end of the shaft where the actuator rod engages the center portion, the actuator rod being configured to move the first and second clamps from a first position to a second position.

9. The system as in claim 1, wherein a first plane and a second plane each bisect the center portion, the first plane being orthogonal to the second plane and intersecting the first and second clamps, the second plane being located equidistant from each of the first and second clamps.

10. The system as in claim 9, wherein the first lens assembly is positioned adjacent to the center portion such that the first plane intersects the first lens assembly.

11. The system as in claim 9, wherein the first lens assembly is positioned adjacent to the center portion such that the second plane intersects the first lens assembly.

12. The system as in claim 9, wherein the first and second planes define four quadrants arranged about the center portion, the first lens assembly being positioned adjacent to the center portion and within one of the four quadrants.

13. The system as in claim 9, wherein the first clamp includes a first proximal element and a first distal element, and the second clamp includes a second proximal element and a second distal element.

14. The system as in claim 13, wherein the first imaging probe extends distally along center portion and proximally into a space between the first proximal element and the first distal element of the first clamp such that the first lens assembly is positioned within the space.

15. The system as in claim 14, wherein the first distal element includes a crossbar extending across at least a portion of an engagement surface thereof, the first imaging probe extending between the crossbar and the engagement surface.

16. The system as in claim 15, wherein the first lens assembly is positioned one of proximal to the crossbar and distal to the crossbar.

17. The system as in claim 13, wherein the first imaging probe extends distally along the center portion and proximally along a proximal side of the proximal element, the first imaging probe being connected to the first side of the proximal element.

18. The system as in claim 13, further comprising a second OCT catheter having a second imaging probe and a second lens assembly, the second imaging probe extending from the shaft of the delivery device such that the second lens assembly is positioned between the first and second lateral extents.

19. The system as in claim 1, wherein each of the first and second clamps have a first end defining an opening for receipt of a leaflet into the clamp, a second end defining a closed end of the clamp, and a length extending between the first and second ends defining a maximum leaflet insertion depth.

20. The system as in claim 19, wherein the lens assembly is positioned relative to first and second clamps such that light emitted therefrom intersects 50% of the maximum leaflet insertion depth of the first and second clamps.
